SEQUENCE LISTING (15 sequences)

SEQ ID NO: 1 - protein, 1195 aa, synthetic construct
  REGION 1..1195: note = S-2P W recombinant protein
QCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQ

SEQ ID NO: 2 - protein, 32 aa, synthetic construct
  REGION 1..32: note = T4 fibritin trimerization domain
GSGYIPEAPRDGQAYVRKDGEWVLLSTFLGRS

SEQ ID NO: 3 - protein, 9 aa, synthetic construct
  REGION 1..9: note = HRV3C protease cleavage site
LEVLFQGPG

SEQ ID NO: 4 - protein, 30 aa, synthetic construct
  REGION 1..30: note = Twin-Strep Tag
SAWSHPQFEKGGGSGGGSGGSAWSHPQFEK

SEQ ID NO: 5 - protein, 1227 aa, synthetic construct
  REGION 1..1227: note = S-2P Beta recombinant protein
QCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRS

SEQ ID NO: 6 - protein, 1233 aa, synthetic construct
  REGION 1..1233: note = S-2P W recombinant protein
QCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQ

SEQ ID NO: 7 - DNA, 22 bp, synthetic construct
  misc_feature 1..22: note = CpG motif
  source 1..22: mol_type = other DNA
tgactgtgaacgttcgagatga

SEQ ID NO: 8 - DNA, 24 bp, synthetic construct
  misc_feature 1..24: note = CpG motif
  source 1..24: mol_type = other DNA
tcgtcgttttgtcgttttgtcgtt

SEQ ID NO: 9 - DNA, 20 bp, synthetic construct
  misc_feature 1..20: note = CpG motif
  source 1..20: mol_type = other DNA
ggtgcatcgatgcagggggg

SEQ ID NO: 10 - DNA, 22 bp, synthetic construct
  misc_feature 1..22: note = CpG motif
  source 1..22: mol_type = other DNA
tccatggacgttcctgagcgtt

SEQ ID NO: 11 - DNA, 22 bp, synthetic construct
  misc_feature 1..22: note = CpG motif
  source 1..22: mol_type = other DNA
tcgtcgttcgaacgacgttgat

SEQ ID NO: 12 - DNA, 23 bp, synthetic construct
  misc_feature 1..23: note = CpG motif
  source 1..23: mol_type = other DNA
tcgtcgacgatcggcgcgcgccg

SEQ ID NO: 13 - protein, 1192 aa, synthetic construct
  REGION 1..1192: note = S-2P Beta recombinant protein
QCVNFTTRTQLPPAYTNSFTRGVYYPDKVFRSSVLHSTQDLFLPFFSNVTWFHAIHVSGTNGTKRFANPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRGLPQGFSALEPLVDLPIGINITRFQTLHISYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGSASSVASQSIIAYTMSLGVENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQ

SEQ ID NO: 14 - protein, 1224 aa, synthetic construct
  REGION 1..1224: note = S-2P Beta recombinant protein
QCVNFTTRTQLPPAYTNSFTRGVYYPDKVFRSSVLHSTQDLFLPFFSNVTWFHAIHVSGTNGTKRFANPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRGLPQGFSALEPLVDLPIGINITRFQTLHISYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGSASSVASQSIIAYTMSLGVENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRS

SEQ ID NO: 15 - protein, 1230 aa, synthetic construct
  REGION 1..1230: note = S-2P Beta recombinant protein
QCVNFTTRTQLPPAYTNSFTRGVYYPDKVFRSSVLHSTQDLFLPFFSNVTWFHAIHVSGTNGTKRFANPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRGLPQGFSALEPLVDLPIGINITRFQTLHISYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGSASSVASQSIIAYTMSLGVENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQ

(19) United States
(12) Patent Application Publication
(10) Pub. No.: US 2024/0197862 A1
(43) Pub. Date: Jun. 20, 2024

(54) IMMUNOGENIC COMPOSITIONS AND METHODS FOR IMMUNIZATION AGAINST VARIANTS OF SEVERE ACUTE RESPIRATORY SYNDROME CORONAVIRUS 2 (SARS-COV-2)

(71) Applicants: Medigen Vaccine Biologics Corporation, Zhubei City (TW); Dynavax Technologies Corporation, Emeryville, CA (US)

(72) Inventors: Charles CHEN, Taipei (TW); Tsun-Yung Kuo, I-Lan County (TW); Chung-Chin Wu, I-Lan County (TW); Wei-Hsuan Tang, I-Lan County (TW); Chia-En Lien, Taipei (TW); Yi-Jiun Lin, Taipei (TW); Meei-Yun Lin, Taipei (TW)

(21) Appl. No.: 18/592,811

(22) Filed: Mar. 1, 2024

Related U.S. Application Data
(63) Continuation of application No. PCT/CN2022/116668, filed on Sep. 2, 2022.
(60) Provisional application No. 63/330,114, filed on Apr. 12, 2022.
(60) Provisional application No. 63/296,193, filed on Jan. 4, 2022.
(60) Provisional application No. 63/251,741, filed on Oct. 4, 2021.
(60) Provisional application No. 63/248,189, filed on Sep. 24, 2021.
(60) Provisional application No. 63/240,080, filed on Sep. 2, 2021.

(51) Int. Cl. A61K 39/215; A61K 39/00; A61P 37/04; C12N 7/00

(52) U.S. Cl. CPC A61K 39/215; A61P 37/04; C12N 7/00; A61K 2039/54; A61K 2039/545; A61K 2039/55505; A61K 2039/55561; C12N 2770/20022; C12N 2770/20034; C12N 2770/20071

(57) ABSTRACT

The present invention relates to immunogenic compositions and methods for immunization against variants of severe acute respiratory syndrome coronavirus 2 (SARS-COV-2), especially to an immunogenic composition having a recombinant SARS-CoV-2 S protein derived from Beta (B.1.351) variant and methods using an immunogenic composition derived from SARS-COV-2 Beta (B.1.351) variant.

### CROSS-REFERENCE TO RELATED APPLICATIONS

This application is a continuation application of International Patent Application No. PCT/CN2022/116668, filed on Sep. 2, 2022, which itself claims priority to and the benefit of U.S. Provisional Application No. 63/240,080, filed on Sep. 2, 2021, No. 63/248,189, filed on Sep. 24, 2021, No. 63/251,741, filed on Oct. 4, 2021, No. 63/296, 193, filed on Jan. 4, 2022, and No. 63/330, 114, filed on Apr. 12, 2022, the disclosure of which is incorporated by reference in its entirety.

The sequence information contained in the Sequence Listing XML file, with the file name “P24-0018US_SequenceListing.xml” created on Feb. 22, 2024 and having a file size of 27,254 bytes, is incorporated by reference herein in its entirety.

### BACKGROUND OF THE INVENTION

### 1. Field of the Invention

The present invention relates to immunogenic compositions and methods for immunization against variants of severe acute respiratory syndrome coronavirus 2 (SARS-COV-2), especially to an immunogenic composition having a recombinant SARS-CoV-2 S protein derived from Beta (B.1.351) variant and methods using the immunogenic composition derived from SARS-COV-2 Beta (B.1.351) variant.

### 2. Description of the Prior Art

In the end of 2019, the World Health Organization (WHO) was alerted to several cases of pneumonia caused by a novel coronavirus. The viral pathogen did not match any other known virus and was later officially named “severe acute respiratory syndrome coronavirus 2 (SARS-COV-2).” The official name of the disease caused by SARS-COV-2 is coronavirus disease 2019 (COVID-19). Common symptoms of COVID-19 include fever, dry cough, fatigue, tiredness, muscle or body aches, sore throat, diarrhea, conjunctivitis, headache, loss of taste or smell, a rash on skin, and shortness of breath. While the majority of cases result in mild symptoms, some progress to acute respiratory distress syndrome (ARDS), precipitated by cytokine storm, multi-organ failure, septic shock, and blood clots. The first confirmed death from the coronavirus infection occurred on Jan. 9, 2020, and as of Aug. 22, 2022, 593,269,262 confirmed cases of COVID-19, including 6,446,547 deaths, have been reported to the WHO (WHO Coronavirus (COVID-19) Dashboard, https://covid19.who.int). The numbers are still growing fast.

Since the beginning of the COVID-19 pandemic, mutants have been detected periodically. A number of them were found to carry mutations in the crucial receptor-binding domain (RBD), a prime target for antibody recognition and neutralization. The most representative of these variants are B.1.1.7 (Alpha variant), B.1.351 (Bata variant), B.1.617.2 or AY.1 (Delta variant), P.1 (Gamma variant), and Omicron (B.1.1.529). The variants with these mutations were found to decrease neutralization capabilities of monoclonal antibodies and vaccine-induced antibodies, and this could potentially render current therapeutics and vaccines ineffective (Garcia-Beltran et al., Cell, 184(9):2372-2383.e9, 2021) and cause cases of vaccine breakthrough infections. Therefore, methods that are effective against highly infectious variants of SARS-COV-2 are urgently needed.

### SUMMARY OF THE INVENTION

The present invention relates to an immunogenic composition against a severe acute respiratory syndrome coronavirus 2 (SARS-COV-2), comprising an antigenic recombinant protein and an adjuvant selected from the group consisting of an aluminum-containing adjuvant, an unmethylated cytosine-phosphate-guanosine (CpG) motif, and a combination thereof, wherein the antigenic recombinant protein substantially consists of residues 14-1205 of spike protein of SARS-COV-2 Beta variant with proline substitutions at residues 983 and 984 and a “GSAS” substitution at residues 679-682 and a C-terminal T4 fibritin trimerization domain.

In some embodiments, the immunogenic composition described herein provides one or more of improved immunogenicity, an enhanced immune response, and/or broad-spectrum immunity.

In other embodiments, methods, formulations, articles, devices, and/or preparations for administering the immunogenic composition described herein, which provides improved immunogenicity, an enhanced immune response, and/or a broad-spectrum immunity, to a subject are also disclosed.

Those skilled in the art will recognize, or be able to ascertain using no more

than routine experimentation, many equivalents to the specific embodiments of the invention described herein. Such equivalents are intended to be encompassed by the following embodiments.

Embodiment 1. An immunogenic composition against a severe acute respiratory syndrome coronavirus 2 (SARS-COV-2), comprising an antigenic recombinant protein and an adjuvant selected from the group consisting of an aluminum-containing adjuvant, an unmethylated cytosine-phosphate-guanosine (CpG) motif, and a combination thereof, wherein the antigenic recombinant protein substantially consists of residues 14-1205 of spike protein of SARS-COV-2 Beta variant with proline substitutions at residues 983 and 984 and a “GSAS” substitution at residues 679-682 and a C-terminal T4 fibritin trimerization domain.

Embodiment 2. The immunogenic composition of Embodiment 1, wherein the residues 14-1205 of spike protein of SARS-COV-2 Beta variant with proline substitutions at residues 983 and 984 and a “GSAS” substitution at residues 679-682 comprise an amino acid sequence of SEQ ID NO: 13 or the amino acid sequence at least 90%, 95%, 96%, 97%, 98%, or 99% to SEQ ID NO: 13.

Embodiment 3. The immunogenic composition of Embodiment 1 or 2, wherein the C-terminal T4 fibritin trimerization motif comprises an amino acid sequence of SEQ ID NO: 2 or the amino acid sequence at least 90%, 95%, 96%, 97%, 98%, or 99% to SEQ ID NO: 2.

Embodiment 4. The immunogenic composition of any one of Embodiments 1 to 3, wherein the antigenic recombinant protein comprises an amino acid sequence of SEQ ID NO: 14 or 15, or the amino acid sequence at least 95%, 96%, 97%, 98%, or 99% identical to SEQ ID NO: 14 or 15.

Embodiment 5. The immunogenic composition of any one of Embodiments 1 to 4, wherein the aluminum-containing adjuvant comprises aluminum hydroxide, aluminum oxyhydroxide, aluminum hydroxide gel, aluminum phosphate, aluminum phosphate gel, aluminum hydroxyphosphate, aluminum hydroxyphosphate sulfate, amorphous aluminum hydroxyphosphate sulfate, potassium aluminum sulfate, aluminum monostearate or a combination thereof.

Embodiment 6. The immunogenic composition of any one of Embodiments 1 to 5, wherein a 0.5 ml dose of the immunogenic composition comprises from about 250 to about 1500 μg Al³⁺, or about 375 μg Al³⁺ or about 750 μg Al³⁺.

Embodiment 7. The immunogenic composition of any one of Embodiments 1 to 6, wherein the unmethylated CpG motif comprises a synthetic oligodeoxynucleotide (ODN) of SEQ ID NO: 7, SEQ ID NO: 8, SEQ ID NO: 9, SEQ ID NO: 10, SEQ ID NO: 11, SEQ ID NO: 12, or a combination thereof.

Embodiment 8. The immunogenic composition of any one of Embodiments 1 to 7, wherein a 0.5 ml dose of the immunogenic composition comprises from about 750 to about 3000 μg of the unmethylated CpG motif, or about 750 μg, 1500 μg, or 3000 μg of the unmethylated CpG motif.

Embodiment 9. The immunogenic composition of any one of Embodiments 1 to 8, wherein the immunogenic composition can be stored at 40° C. to 42ºC for 3 to 7 days.

Embodiment 10. A method for eliciting an immune response against a severe acute respiratory syndrome coronavirus 2 (SARS-COV-2) in a subject in need thereof, comprising administering to the subject at least one dose of an immunogenic composition of any one of Embodiments 1 to 9.

Embodiment 11. A method for protecting a subject in need thereof from infection with a severe acute respiratory syndrome coronavirus 2 (SARS-COV-2), comprising administering to the subject at least one dose of an immunogenic composition of any one of Embodiments 1 to 9.

Embodiment 12. A method for preventing a subject in need thereof from contracting COVID-19 disease, comprising administering to the subject at least one dose of an immunogenic composition of any one of Embodiments 1 to 9, wherein the COVID-19 disease is caused by a severe acute respiratory syndrome coronavirus 2 (SARS-COV-2).

Embodiment 13. Use of the immunogenic composition of any one of Embodiments 1 to 9 for eliciting an immune response against a severe acute respiratory syndrome coronavirus 2 (SARS-COV-2) in a subject in need thereof.

Embodiment 14. Use of the immunogenic composition of any one of Embodiments 1 to 9 for protecting a subject in need thereof from infection with severe acute respiratory syndrome coronavirus 2 (SARS-COV-2).

Embodiment 15. Use of the immunogenic composition of any one of Embodiments 1 to 9 for preventing a subject in need thereof from contracting COVID-19 disease, wherein the COVID-19 disease is caused by a severe acute respiratory syndrome coronavirus 2 (SARS-COV-2).

Embodiment 16. Use of the immunogenic composition of any one of Embodiments 1 to 9 for manufacturing a medicament for eliciting an immune response against a severe acute respiratory syndrome coronavirus 2 (SARS-COV-2) in a subject in need thereof.

Embodiment 17. Use of the immunogenic composition of any one of Embodiments 1 to 9 for manufacturing a medicament for protecting a subject in need thereof from infection with severe acute respiratory syndrome coronavirus 2 (SARS-CoV-2).

Embodiment 18. Use of the immunogenic composition of any one of Embodiments 1 to 9 for manufacturing a medicament for preventing a subject in need thereof from contracting COVID-19 disease, wherein the COVID-19 disease is caused by a severe acute respiratory syndrome coronavirus 2 (SARS-COV-2).

Embodiment 19. The use of any one of Embodiments 13 to 18, wherein at least one dose of the immunogenic composition of any one of Embodiments 1 to 9 is administered to the subject.

Embodiment 20. The method of Embodiment 10 or the use of Embodiment 13 or 16, wherein the immune response comprises production of neutralizing antibodies against SARS-COV-2 and Th1-skewed immune response.

Embodiment 21. The method or the use of any one of Embodiments 10 to 19, wherein the subject is administered with a first dose and a second dose of the immunogenic composition of any one of Embodiments 1 to 9 with a suitable interval between the first dose and the second dose.

Embodiment 22. The method or the use of any one of Embodiments 10 to 19, wherein the subject is administered with a first dose, a second dose, and a third dose of the immunogenic composition of any one of Embodiments 1 to 9 with a first suitable interval between the first dose and the second dose, and with a second suitable interval between the second dose and the third dose.

Embodiment 23. The method or the use of any one of Embodiments 10 to 19, wherein the subject is administered with a first dose of an immunogenic composition derived from SARS-COV-2 WT strain (wild type), and a second dose of the immunogenic composition of any one of Embodiments 1 to 9 with a suitable interval between the first dose and the second dose.

Embodiment 24. The method or the use of any one of Embodiments 10 to 19, wherein the subject is administered with a first dose and a second dose of an immunogenic composition derived from SARS-COV-2 WT strain, and a third dose of the immunogenic composition of any one of Embodiments 1 to 9 with a first suitable interval between the first dose and the second dose, and with a second suitable interval between the second dose and the third dose.

Embodiment 25. The method or the use of any one of Embodiments 10 to 19, wherein the subject is administered with a first dose, a second dose, and a third dose of an immunogenic composition derived from SARS-COV-2 WT strain, and a fourth dose of the immunogenic composition of any one of Embodiments 1 to 9 with a first suitable interval between the first dose and the second dose, a second suitable interval between the second dose and the third dose, and a third suitable interval between the third dose and the fourth dose.

Embodiment 26. The method or the use of any one of Embodiments 10 to 19, wherein the subject is administered with a first dose of an immunogenic composition derived from SARS-COV-2 WT strain, and a second dose and a third dose of the immunogenic composition of any one of Embodiments 1 to 9 with a first suitable interval between the first dose and the second dose, and with a second suitable interval between the second dose and the third dose.

Embodiment 27. The method or the use of any one of Embodiments 23 to 26, wherein the immunogenic composition derived from SARS-COV-2 WT strain comprises a polypeptide sequence of at least a fragment of spike protein of SARS-COV-2 WT strain.

Embodiment 28. The method or the use of any one of Embodiments 23 to 26, wherein the immunogenic composition derived from SARS-COV-2 WT strain comprises a polynucleotide sequence encoding at least a fragment of spike protein of SARS-COV-2 WT strain.

Embodiment 29. The method or the use of any one of Embodiments 23 to 28, wherein the at least a fragment of spike protein of SARS-COV-2 WT strain substantially consists of residues 14-1208 of spike protein of SARS-COV-2 WT strain with proline substitutions at residues 986 and 987 and a “GSAS” substitution at residues 682-685 and a C-terminal T4 fibritin trimerization domain.

Embodiment 30. The method or the use of Embodiment 29, wherein the residues 14-1208 of spike protein with proline substitutions at residues 986 and 987 and a “GSAS” substitution at residues 682-685 comprise an amino acid sequence of SEQ ID NO: 1 or the amino acid sequence at least 90%, 95%, 96%, 97%, 98%, or 99% to SEQ ID NO: 1.

Embodiment 31. The method or the use of Embodiments 29 or 30, wherein the C-terminal T4 fibritin trimerization motif comprises an amino acid sequence of SEQ ID NO: 2 or the amino acid sequence at least 90%, 95%, 96%, 97%, 98%, or 99% to SEQ ID NO: 2.

Embodiment 32. The method or the use of any one of Embodiments 23 to 31, wherein the at least a fragment of spike protein of SARS-COV-2 WT strain comprises an amino acid sequence of SEQ ID NO: 5 or 6 or the amino acid sequence at least 90%, 95%, 96%, 97%, 98%, or 99% to SEQ ID NO: 5 or 6.

Embodiment 33. The method or the use of any one of Embodiments 23 to 27, wherein the immunogenic composition derived from SARS-COV-2 WT strain comprises a polypeptide sequence of SEQ ID NO: 5 or 6 or the polypeptide sequence at least 90%, 95%, 96%, 97%, 98%, or 99% identical to SEQ ID NO: 5 or 6, and an adjuvant selected from the group consisting of an aluminum-containing adjuvant, an unmethylated cytosine-phosphate-guanosine (CpG) motif, and a combination thereof.

Embodiment 34. The method or the use of Embodiment 33, wherein the aluminum-containing adjuvant comprises aluminum hydroxide, aluminum oxyhydroxide, aluminum hydroxide gel, aluminum phosphate, aluminum phosphate gel, aluminum hydroxyphosphate, aluminum hydroxyphosphate sulfate, amorphous aluminum hydroxyphosphate sulfate, potassium aluminum sulfate, aluminum monostearate or a combination thereof.

Embodiment 35. The method or the use of Embodiment 33 or 34, wherein a 0.5 ml dose of the immunogenic composition derived from SARS-COV-2 WT strain comprises from about 250 to about 500 μg Al³⁺, or about 375 μg Al³⁺.

Embodiment 36. The method or the use of any one of Embodiments 33 to 35, wherein the unmethylated CpG motif comprises a synthetic oligodeoxynucleotide (ODN) of SEQ ID NO: 8, SEQ ID NO: 9, SEQ ID NO: 10, SEQ ID NO: 11, SEQ ID NO: 12, SEQ ID NO: 13, or a combination thereof.

Embodiment 37. The method or the use of any one of Embodiments 33 to 36, wherein a 0.5 ml dose of the immunogenic composition derived from SARS-COV-2 WT strain comprises from about 750 to about 3000 μg of the unmethylated CpG motif, or wherein the immunogenic composition comprises about 750 μg, about 1500 μg, or about 3000 μg of the unmethylated CpG motif.

Embodiment 38. The method or the use of any one of Embodiments 10 to 37, wherein the severe acute respiratory syndrome coronavirus 2 (SARS-COV-2) is a wild type strain or a variant.

Embodiment 39. The method or the use of any one of Embodiments 10 to 38, wherein the subject is administered by intramuscular injection.

These and other aspects will become apparent from the following description of the preferred embodiment taken in conjunction with the following drawings.

### BRIEF DESCRIPTION OF THE DRAWINGS

The accompanying drawings illustrate one or more embodiments of the invention and, together with the written description, serve to explain the principles of the invention. Wherever possible, the same reference numbers are used throughout the drawings to refer to the same or like elements of an embodiment.

FIGS. 1A to 1E show summary of live virus neutralization assay in Syrian hamster model 5 weeks after the last (either second or third) immunization. Hamsters (N=10 per group) were immunized twice at 3 weeks apart with two doses of S-2P W recombinant protein (Group 1, W+W), or immunized three times at 3 weeks apart with three doses of S-2P W recombinant protein (Group 2, W+W+W) or two doses of S-2P W recombinant protein followed by one dose of S-2P Beta recombinant protein (Group 3, W+W+B), or with adjuvant alone (Group 4). The antisera were harvested at 5 weeks (day 78) after the last injection and subjected to live virus neutralization assay with SARS-CoV-2 WT strain (wild type, FIG. 1A), the Alpha variant (FIG. 1B), the Beta variant (FIG. 1C), the Gamma variant (FIG. 1D), and the Delta variant (FIG. 1E). Each dot represents individual serum sample neutralizing titer (NT₅₀). Bars indicate geometric mean titers (GMT) and error bars indicate 95% confidence intervals, and statistical significance was calculated with Mann-Whitney test. Dotted lines represent lower limits of detection (200 in NT₅₀). *p<0.05, **p<0.01, *** p<0.001, ****p<0.0001, and ns means no statistically significant difference.

FIG. 2 shows summary of pseudovirus-based neutralization assays in Syrian hamster model 5 weeks after the last (either second or third) immunization. The antisera were collected as described in FIGS. 1A to 1E (N=10 per group) and subjected to neutralization assays with pseudoviruses of SARS-COV-2 WT strain (wild type), B.1.1.7 (Alpha), B.1.351 (Beta), B.1.617.2 (Delta), AY.1 (Delta), C.37 (Lambda), and B.1.621 (Mu) variants. Each dot represents individual serum sample neutralizing titer. Results are presented as geometric mean with error bars representing 95% confidence interval. The lower dotted line represents the lower limit of detection (200 in NT₅₀), and the upper dotted line represents the high limit of detection (25600 in NT₅₀).

FIG. 3 shows body weight change of hamsters within 6 days post infection (d.p.i.) with SARS-COV-2 Delta variant. Hamsters were immunized as described in FIGS. 1A to 1E (N=10 per group) and challenged with the Delta variant 53 days after completion of immunization (Day 96). Line plots show mean+standard error of the mean (SEM). Statistical significance was calculated with two-way ANOVA and Dunnett test. ****p<0.0001.

FIGS. 4A and 4B show viral load in lungs of hamsters at 3 and 6 days post infection (dpi) with SARS-COV-2 Delta variant. Hamsters were immunized as described in FIGS. 1A to 1E (N=10 per group), challenged with the Delta variant 53 days after completion of immunization (Day 96), and euthanized at 3 or 6 dpi. Lung tissue samples were collected for viral load determination by quantitative PCR of viral genome RNA (FIG. 4A) and TCID₅₀ assay for infectious virus load (FIG. 4B). Results are presented as geometric mean with error bars representing 95% confidence interval and statistical significance calculated with Kruskal-Wallis test with Dunn's test compared with negative control (Group 4). Each dot represents an individual sample virus titer. Dotted lines represent lower limits of detection (L.O.D.). *p<0.05, **p<0.01, ***p<0.001.

FIG. 5 shows lung pathology scoring in hamsters at 3 or 6 days post infection (dpi) with SARS-COV-2 Delta variant. Hamsters were immunized, challenged with the Delta variant, and euthanized as described in FIGS. 4A and 4B. Lung tissue samples were collected for sectioning and staining. The histopathology sections were scored as outlined in the methods and the results tabulated. Each dot represents an individual sample histopathology score. Results are presented as mean of lung pathology scores with error bars representing standard error and statistical significance calculated with Kruskal-Wallis test with Dunn's test. *p<0.05.

FIG. 6 shows correlation between viral RNA and the neutralizing titer (NT₅₀) against SARS-COV-2 Delta variant in Syrian hamster model. The antisera were collected as described in FIGS. 1A to 1E (N=10 per group) and subjected to neutralization assays with SARS-COV-2 Delta variant. Lung tissue samples were collected at 3 dpi for viral

RNA determination as described in FIGS. 4A and 4B. The line is prediction of viral RNA in lungs on the neutralizing titer (NT₅₀) against SARS-COV-2 Delta variant in antisera. Each hollow circle represents an individual sample. The 95% confidence band of the fitted values is shown by the grey area.

FIG. 7 shows summary of pseudovirus-based neutralization assays in Syrian hamster model 5 weeks after the third immunization. Hamsters (N=10 per group) were immunized three times at 3 weeks apart with three doses of S-2P W recombinant protein (Group 1, W+W+W) or two doses of S-2P W recombinant protein followed by one dose of S-2P Beta recombinant protein (Group 2, W+W+B). The antisera were harvested at 5 weeks (day 78) after the last injection and subjected to neutralization assays with pseudoviruses of SARS-COV-2 B.1.1.529 (Omicron) variant. Each dot represents the neutralizing titer of a mixture of 2 serum samples. Results are presented as geometric mean with error bars representing 95% confidence interval. The lower dotted line represents the lower limit of detection (100 in ID₅₀), and the upper dotted line represents the high limit of detection (12800 in ID₅₀). Statistical significance was calculated with

Mann-Whitney test. **p<0.01.

FIG. 8 shows summary of pseudovirus-based neutralization assays in BALB/c mice 2 weeks after the second immunization. BALB/c mice (N=5 per group) were immunized twice at 3 weeks apart with two doses of S-2P Beta recombinant protein stored at 40° C. for 3 days (Group 1), stored at 40° C. for 7 days (Group 2), stored at 42° C. for 3 days (Group 3), stored at 42° C. for 7 days (Group 4), or constantly stored at 4° C. (Group 5, control group). The antisera were harvested at 2 weeks after the second injection and subjected to neutralization assays with pseudoviruses of SARS-COV-2 Beta variant (B.1.351). Each dot represents individual serum sample neutralizing titer. Bars indicate geometric mean titers (GMT) of 90% inhibition dilution (ID₉₀) and error bars indicate 95% confidence intervals. The lower dotted line represents the lower limit of detection (200 in ID₉₀), the upper dotted line represents the upper limit of detection (25600 in ID₉₀). Statistical significance was calculated with Mann-Whitney test, and ns means no statistically significant difference.

FIGS. 9A to 9D show summary of pseudovirus-based neutralization assays in BALB/c mice 2 weeks after the second immunization. BALB/c mice (N=5 per group) were immunized twice at 3 weeks apart with two doses of S-2P W recombinant protein (Group 1, W+W), two doses of S-2P Beta recombinant protein (Group 2, B+B), one dose of S-2P W recombinant protein followed by one dose of S-2P Beta recombinant protein (Group 3, W+B), three doses of S-2P W recombinant protein (Group 4, W+W+W), two doses of S-2P W recombinant protein followed by one dose of S-2P Beta recombinant protein (Group 5, W+W+B), three doses of S-2P Beta recombinant protein (Group 6, B+B+B), or with adjuvant alone (Group 7). The antisera were harvested at 2 weeks after the second injection and subjected to neutralization assays with pseudoviruses of SARS-CoV-2 WT strain (WT, FIG. 9A), the Beta variant (B.1.351, FIG. 9B), the Delta variant (B.1.617.2, FIG. 9C), and the Omicron variant (B.1.1.529/BA.1, FIG. 9D). Each dot represents individual serum sample neutralizing titer. Bars indicate geometric mean titers (GMT) of 50% inhibition dilution (ID₅₀) and error bars indicate 95% confidence intervals. The lower dotted line represents the lower limit of detection (150 in ID₅₀), and the upper dotted line represents the high limit of detection (19200 in ID₅₀). Statistical significance was calculated with Mann-Whitney test. *p<0.05, **p<0.01.

FIG. 10 shows summary of solicited adverse events in a Phase I clinical trial. Participants were asked to record solicited local and systemic adverse events in the participant's diary card for up to 7 days after the booster vaccination. Solicited adverse events (AEs) were tabulated and graded as mild, moderate, or severe.

FIGS. 11A and 11B show summary of live virus neutralization assay in the Phase I clinical trial. Participants vaccinated with 2 prior doses of MVC-COV1901 received a booster dose of MVC-COV1901 (Subgroup A-1; W+W+W), 15 μg MVC-COV1901-Beta (Subgroup A-2; W+W+15B), or 25 μg MVC-COV1901-Beta (Subgroup A-3; W+W+25B) (FIG. 11A), and participants vaccinated with 3 prior doses of MVC-COV1901 also received a booster dose of MVC-COV1901 (Subgroup B-1; W+W+W+W), 15 μg MVC-COV1901-Beta (Subgroup B-2; W+W+W+15B), or 25 μg MVC-COV1901-Beta (Subgroup B-3; W+W+W+25B) (FIG. 11B). Sera of participants were collected at Visits 2 (the day of the booster dose; baseline) and 5 (4 weeks after the booster dose) and subjected to live virus neutralization assay with wild type and Beta variant SARS-COV-2. Bars indicate GMT of 50% neutralization titer (NT₅₀) and error bars indicate 95% confidence intervals, and statistical significance was calculated with Kruskal-Wallis with corrected Dunn's multiple comparisons test. *p<0.05.

FIG. 12 shows summary of anti-spike immunoglobulin G (IgG) titration in the Phase I clinical trial. Sera were collected as described in FIGS. 11A and 11B at Visits 2, 4 (2 weeks after the booster dose), and 5 and subjected to anti-spike IgG titration. Bars indicate GMT and error bars indicate 95% confidence intervals, and statistical significance was calculated with Kruskal-Wallis with corrected Dunn's multiple comparisons test.

FIG. 13 shows summary of pseudovirus-based neutralization assays in the Phase I clinical trial. Sera were collected as described in FIGS. 11A and 11B at Visits 2 and 4 and subjected to neutralization assays with pseudoviruses of SARS-COV-2 wild type and Omicron variant (BA.4/BA.5). Bars indicate GMT of 50% inhibition dilution (ID₅₀) and error bars indicate 95% confidence intervals. The lower dotted line represents the lower limit of detection (20 in ID₅₀), and the upper dotted line represents the high limit of detection (2560 in ID₅₀). Statistical significance was calculated with Kruskal-Wallis with corrected Dunn's multiple comparisons test. **p<0.01.

### DETAILED DESCRIPTION OF THE PREFERRED EMBODIMENT

The present invention relates to an immunogenic composition against SARS-CoV-2. The immunogenic composition comprises an antigenic recombinant protein and an adjuvant containing an aluminum-containing adjuvant and/or an unmethylated cytosine-phosphate-guanosine (CpG) motif. The antigenic recombinant protein comprises residues 14-1205 of spike protein of SARS-COV-2 Beta variant with proline substitutions at residues 983 and 984 and a “GSAS” substitution at residues 679-682 and a C-terminal T4 fibritin trimerization domain. In some embodiments, the antigenic recombinant protein comprises an amino acid sequence of SEQ ID NO: 14 or 15, or the amino acid sequence at least 95%, 96%, 97%, 98%, or 99% identical to SEQ ID NO: 14 or 15.

The present invention further relates to a method for eliciting an immune response against severe acute respiratory syndrome coronavirus 2 (SARS-COV-2) in a subject in need thereof, a method for protecting a subject in need thereof from infection with severe acute respiratory syndrome coronavirus 2 (SARS-COV-2), and a method for preventing a subject in need thereof from contracting COVID-19 disease. The methods comprise administering to the subject at least one dose of an immunogenic composition comprising an antigenic recombinant protein and an adjuvant selected from the group consisting of an aluminum-containing adjuvant, an unmethylated cytosine-phosphate-guanosine (CpG) motif, and a combination thereof, wherein the antigenic recombinant protein substantially consists of residues 14-1205 of spike protein of SARS-COV-2 Beta variant with proline substitutions at residues 983 and 984 and a “GSAS” substitution at residues 679-682 and a C-terminal T4 fibritin trimerization domain.

The present invention also relates to use of the immunogenic composition of the present invention for eliciting an immune response against a severe acute respiratory syndrome coronavirus 2 (SARS-COV-2) in a subject in need thereof, for protecting a subject in need thereof from infection with severe acute respiratory syndrome coronavirus 2 (SARS-COV-2), and for preventing a subject in need thereof from contracting COVID-19 disease caused by SARS-COV-2.

### Definitions

All scientific and technical terms used herein, unless otherwise defined below, are intended to have the same meaning as commonly understood by one of ordinary skill in the art. References to techniques employed herein are intended to refer to the techniques as commonly understood in the art, including variations on those techniques or substitutions of equivalent or later-developed techniques which would be apparent to one of skill in the art. In addition, in order to more clearly and concisely describe the subject matter which is the invention, the following definitions are provided for certain terms which are used in the specification and appended claims.

As used herein, the singular form “a”, “an”, and “the” includes plural

references unless indicated otherwise. For example, “an” excipient includes one or more excipients.

As used herein, the term “about” means+/−10% of the recited value. The phrase “and/or,” as used herein in the specification and in the claims,

should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

The phrase “comprising” as used herein is open-ended, indicating that such embodiments may include additional elements. In contrast, the phrase “consisting of” is closed, indicating that such embodiments do not include additional elements (except for trace impurities). The phrase “consisting essentially of” is partially closed, indicating that such embodiments may further comprise elements that do not materially change the basic characteristics of such embodiments.

It should also be understood that, unless clearly indicated to the contrary, in any methods claimed herein that include more than one step or act, the order of the steps or acts of the method is not necessarily limited to the order in which the steps or acts of the method are recited.

As used interchangeably herein, the terms “polynucleotide” and “oligonucleotide” include single-stranded DNA (ssDNA), double-stranded DNA (dsDNA), single-stranded RNA (ssRNA) and double-stranded RNA (dsRNA), modified oligonucleotides and oligonucleosides or combinations thereof. The oligonucleotide can be linearly or circularly configured, or the oligonucleotide can contain both linear and circular segments. Oligonucleotides are polymers of nucleosides joined, generally, through phosphodiester linkages, although alternate linkages, such as phosphorothioate esters may also be used in oligonucleotides. A nucleoside consists of a purine (adenine (A) or guanine (G) or derivative thereof) or pyrimidine (thymine (T), cytosine (C) or uracil (U), or derivative thereof) base bonded to a sugar. The four nucleoside units (or bases) in DNA are called deoxyadenosine, deoxyguanosine, thymidine, and deoxycytidine. A nucleotide is a phosphate ester of a nucleoside.

As used herein, the term “severe acute respiratory syndrome coronavirus 2 (SARS-COV-2)” refers to the strains and variants of coronavirus that cause coronavirus disease 2019 (COVID-19). SARS-COV-2 is a positive-sense single-stranded RNA virus that is a member of the genus Betacoronavirus of the family Coronavirinae. The RNA sequence of SARS-COV-2 is approximately 30,000 bases in length. Each SARS-COV-2 virion is 50-200 nanometres in diameter. Like other coronaviruses, SARS-COV-2 has four structural proteins, known as the S (spike), E (envelope), M (membrane), and N (nucleocapsid) proteins. The N protein holds the RNA genome, and the S, E, and M proteins together create the viral envelope. The S protein is the protein responsible for allowing the virus to attach to and fuse with the membrane of a host cell.

As used herein, the term “spike protein,” “S polypeptide,” “S protein,” “SARS-CoV-2 spike,” or “SARS-COV-2 S protein,” which can be used interchangeably, refers to a surface structure glycoprotein on SARS COV-2 and is responsible for allowing the virus to attach to and fuse with the membrane of a host cell. Each monomer of trimeric S protein is about 180 kDa, and contains two subunits, S1 and S2, mediating attachment and membrane fusion, respectively. Spike protein mainly enters human cells by binding to the receptor angiotensin converting enzyme 2 (ACE2).

Since the beginning of the COVID-19 pandemic, mutants have been detected periodically. The emergence of variants that posed an increased risk to global public health prompted the characterization of specific Variants of Interest (VOIs), Variants of Concern (VOCs), and Variants Under Monitoring (VUMs), in order to prioritize global monitoring and research, and ultimately to inform the ongoing response to the COVID-19 pandemic (https://www.who.int/en/activities/tracking-SARS-COV-2-variants/).

As used herein, the term Variant of Interest (Vol)” refers to a SARS-COV-2 variant with genetic changes that are predicted or known to affect virus characteristics such as transmissibility, disease severity, immune escape, diagnostic or therapeutic escape; and identified to cause significant community transmission or multiple COVID-19 clusters, in multiple countries with increasing relative prevalence alongside increasing number of cases over time, or other apparent epidemiological impacts to suggest an emerging risk to global public health. Given the continuous evolution of the virus that leads to SARS-COV-2 and the constant developments in people's understanding of the impacts of variants, these definitions may be periodically adjusted (https://www.who.int/en/activities/tracking-SARS-COV-2-variants/).

As used herein, the term “Variant of Concern (VoC)” refers to a SARS-COV-2 variant that meets the definition of a VOI and, through a comparative assessment, has been demonstrated to be associated with one or more of the following changes at a degree of global public health significance: (i) increase in transmissibility or detrimental change in COVID-19 epidemiology; or (ii) increase in virulence or change in clinical disease presentation; or (iii) decrease in effectiveness of public health and social measures or available diagnostics, vaccines, therapeutics. Again, given the continuous evolution of the virus that leads to SARS-COV-2 and the constant developments in people's understanding of the impacts of variants, these definitions may be periodically adjusted. Currently (August, 2022) designated VoC by WHO includes Omicron variant (B.1.1.529, BA.1, BA.1.1, BA.2, BA.3, BA.4 and BA.5 lineages). (https://www.who.int/en/activities/tracking-SARS-COV-2-variants/).

As used herein, the term “Variant Under Monitoring (VUM)” refers to a SARS-CoV-2 variant with genetic changes that are suspected to affect virus characteristics with some indication that it may pose a future risk, but evidence of phenotypic or epidemiological impact is currently unclear, requiring enhanced monitoring and repeat assessment pending new evidence. Again, given the continuous evolution of the virus that leads to SARS-COV-2 and the constant developments in people's understanding of the impacts of variants, these definitions may be periodically adjusted.

As used herein, the term “Alpha variant” also known as lineage B.1.1.7, refers

to a variant of SARS-COV-2. The amino acid sequence of S protein of SARS-COV-2 Alpha variant has the following substitutions compared to the S protein of SARS-COV-2 Wuhan-Hu-1 strain (wild type): 69del, 70del, 144del, (E484K*), (S494P*), N501Y, A570D, D614G, P681H, T716I, S982A, D1118H (K1191N*) (https://www.cdc.gov/coronavirus/2019-ncov/variants/variant-info.html) (https://www.acep.org/corona/covid-19-field-guide/characteristics-of-covid-19-variants-and-mutants/characteristics-of-covid-19-variants-and-mutants/).

As used herein, the term “Beta variant” also known as lineage B.1.351, refers to a variant of SARS-COV-2. The amino acid sequence of S protein of SARS-COV-2 Beta variant has the following substitutions compared to the S protein of SARS-COV-2 WT strain (wild type): D80A, D215G, 241del, 242del, 243del, K417N, E484K, N501Y, D614G, A701V (https://www.cdc.gov/coronavirus/2019-ncov/variants/variant-info.html) (https://www.acep.org/corona/covid-19-field-guide/characteristics-of-covid-19-variants-and-mutants/characteristics-of-covid-19-variants-and-mutants/).

As used herein, the term “Gamma variant” also known as lineage P.1, refers to a variant of SARS-COV-2. The amino acid sequence of S protein of SARS-COV-2 Gamma variant has the following substitutions compared to the S protein of SARS-COV-2 WT strain (wild type): L18F, T20N, P26S, D138Y, R190S, K417T, E484K, N501Y, D614G, H655Y, T1027I (https://www.cdc.gov/coronavirus/2019-ncov/variants/variant-info.html) (https://www.acep.org/corona/covid-19-field-guide/characteristics-of-covid-19-variants-and-mutants/characteristics-of-covid-19-variants-and-mutants/).

As used herein, the term “Delta variant” also known as lineage B.1.617.2 and all its AY sublineages, such as AY.1 and AY.2, refers to a variant of SARS-COV-2. The amino acid sequence of S protein of SARS-COV-2 Delta variant has the following substitutions compared to the S protein of SARS-COV-2 WT strain (wild type): T19R, (V70F*), T95I, G142D, E156-, F157-, R158G, (A222V*), (W258L*), (K417N*), L452R, T478K, D614G, P681R, D950N (https://www.cdc.gov/coronavirus/2019-ncov/variants/variant-info.html) (https://www.acep.org/corona/covid-19-field-guide/characteristics-of-covid-19-variants-and-mutants/characteristics-of-covid-19-variants-and-mutants/).

As used herein, the term “Lambda variant” also known as lineage C.37, refers to a variant of SARS-COV-2. The amino acid sequence of S protein of SARS-COV-2 Lambda variant has the following substitutions compared to the S protein of SARS-COV-2 WT strain (wild type): G75V, T76I, R246N, 247-253del, L452Q, F490S, D614G, T859N (https://outbreak.info/situation-reports?pango=C.37).

As used herein, the term “Mu variant” also known as lineage B. 1.621, refers to a variant of SARS-COV-2. The amino acid sequence of S protein of SARS-COV-2 Mu variant has the following substitutions compared to the S protein of SARS-COV-2 WT strain (wild type): T951, Y144S, Y145N, R346K, E484K, N501Y, D614G, P681H, D950N (https://outbreak.info/situation-reports?pango=B.1.621).

As used herein, the term “Omicron variant” also known as the lineage B.1.1.529 and all its sublineages, such as BA.1, BA.1.1, BA.2, BA.3, BA.4, and BA.5, refers to a variant of SARS-COV-2. The amino acid sequence of S protein of SARS-COV-2 Omicron variant (B.1.1.529) has the following substitutions compared to the S protein of SARS-COV-2 WT strain (wild type): A67V, 469-70, T95I, G142D, A143-145, A211, L212I, ins214EPE, G339D, S371L, S373P, S375F, K417N, N440K, G446S, S477N, T478K, E484A, Q493R, G496S, Q498R, N501Y, Y505H, T547K, D614G, H655Y, N679K, P681H, N764K, D796Y, N856K, Q954H, N969K, and L981F; in addition, the amino acid sequence of S protein of SARS-COV-2 Omicron variant (BA.4/BA.5, both possess identical spike protein sequences) has the following substitutions compared to the S protein of SARS-COV-2 WT strain (wild type): T19I, del24-26, A27S, del69-70, G142D, V213G, G339D, S371F, S373P, S375F, T376A, D405N, R408S, K417N, N440K, L452R, S477N, T478K, E484A, F486V, Q498R, N501Y, Y505H, D614G, H655Y, N679K, P681H, N764K, D796Y, Q954H, and N969K (https://www.cdc.gov/coronavirus/2019-ncov/variants/variant-info.html) (https://www.acep.org/corona/covid-19-field-guide/characteristics-of-covid-19-variants-and-mutants/characteristics-of-covid-19-variants-and-mutants/).

As used interchangeably herein, the terms “COVID-19 vaccine” and “immunogenic composition against severe acute respiratory syndrome coronavirus 2 (SARS-COV-2)” refers to a composition for stimulating or eliciting an immune response against a SARS-COV-2. The immune response includes, but not limited to, production of neutralizing antibodies against SARS-COV-2 and Th1-skewed immune response. In some embodiments, the COVID-19 vaccine is a COVID-19 vaccine administrated to a subject via intramuscular injection, and one dose of the COVID-19 vaccine for human contains 5 μg, 15 μg, or 25 μg S-2P recombinant protein adjuvanted with 750 μg CpG 1018 adjuvant and 375 μg (Al³⁺ equivalent to weight) aluminum hydroxide. In some embodiments, one dose of the COVID-19 vaccine for rodents (e.g., mice, rats, and hamsters) contains ⅕ volume of one dose of the COVID-19 vaccine for human.

As used herein, the terms “adjuvant” refers to a substance which, when added to a composition comprising an antigen, nonspecifically enhances or potentiates an immune response to the antigen in the recipient upon exposure.

As used herein, the term “aluminum-containing adjuvant” refers to an adjuvant including aluminum. In some embodiments, the aluminum-containing adjuvant includes, but not limited to, aluminum hydroxide, aluminum oxyhydroxide, aluminum hydroxide gel, aluminum phosphate, aluminum phosphate gel, aluminum hydroxyphosphate, aluminum hydroxyphosphate sulfate, amorphous aluminum hydroxyphosphate sulfate, potassium aluminum sulfate, aluminum monostearate or a combination thereof. In some embodiments, the aluminum-containing adjuvant is an aluminum-containing adjuvant approved for administration to humans by the FDA. In some embodiments, the aluminum-containing adjuvant is an aluminum hydroxide adjuvant approved for administration to humans by the FDA. In some embodiments, the aluminum-containing adjuvant is an aluminum phosphate adjuvant approved for administration to humans by the FDA.

As used herein, the terms “unmethylated cytosine-phosphate-guanosine (CpG) motif” refers to a CpG-containing oligonucleotide in which the C is unmethylated, and which contributes to a measurable immune response as measured in vitro, in vivo, and/or ex vivo. In some embodiments, the CpG-containing oligonucleotide contains palindromic hexamers following the general formula of: 5′-purine-purine-CG-pyrimidine-pyrimidine-3′. In some preferred embodiments, the unmethylated cytosine-phosphate-guanosine (CpG) motif has an oligonucleotide of SEQ ID NO: 8 (5′-TGACTGTGAACGTTCGAGATGA-3′) in which the Cs of the CGs are unmethylated. In some embodiments, the CpG-containing oligonucleotide contains TCG in which the C is unmethylated, and which is from 8 to 100 nucleotides, preferably 8 to 50 nucleotides, or preferably 8 to 25 nucleotides in length. In some preferred embodiments, the unmethylated cytosine-phosphate-guanosine (CpG) motif has an oligonucleotide of SEQ ID NO: 9 (5′-TCGTCGTTTTGTCGTTTTGTCGTT-3′) in which the Cs of the TCGs are unmethylated. Examples of the unmethylated cytosine-phosphate-guanosine (CpG) motif further includes, but not limited to, 5′-GGTGCATCGATGCAGGGG GG-3′ (SEQ ID NO: 10), 5′-TCCATGGACGTTCCTGAGCGTT-3′ (SEQ ID NO: 11), 5′-TCGTCGTTCGAACGACGTTGAT-3′ (SEQ ID NO: 12), and 5′-TCGTCGACGATCGGC GCGCGCCG-3′ (SEQ ID NO: 13). The CpG-containing oligonucleotide described herein are in their pharmaceutically acceptable salt form unless otherwise indicated. In one preferred embodiment, the CpG-containing oligonucleotides are in the sodium salt form.

An “effective amount” or a “sufficient amount” of a substance is that amount sufficient to effect beneficial or desired results, including clinical results, and, as such, an “effective amount” depends upon the context in which it is being applied. In the context of administering an immunogenic composition, an effective amount contains sufficient adjuvant and SARS-COV-2 S-2P Beta recombinant protein to elicit an immune response. An effective amount can be administered in one or more doses.

The terms “individual” and “subject” refer to mammals. “Mammals” include, but are not limited to, humans, non-human primates (e.g., monkeys), farm animals, sport animals, rodents (e.g., mice, rats, and hamsters) and pets (e.g., dogs and cats).

The term “dose” as used herein in reference to an immunogenic composition refers to a measured portion of the immunogenic composition taken by (administered to or received by) a subject at any one time.

The terms “isolated” and “purified” as used herein refers to a material that is removed from at least one component with which it is naturally associated (e.g., removed from its original environment). The term “isolated,” when used in reference to a recombinant protein, refers to a protein that has been removed from the culture medium of the host cell that produced the protein.

“Stimulation” of a response or parameter includes eliciting and/or enhancing that response or parameter when compared to otherwise same conditions except for a parameter of interest, or alternatively, as compared to another condition (e.g., increase in TLR-signaling in the presence of a TLR agonist as compared to the absence of the TLR agonist). For example, “stimulation” of an immune response means an increase in the response. Depending upon the parameter measured, the increase may be from 5-fold to 500-fold or over, or from 5, 10, 50, or 100-fold to 500, 1,000, 5,000, or 10,000-fold.

As used herein the term “immunization” refers to a process that increases a mammalian subject's reaction to antigen and therefore improves its ability to resist or overcome infection.

The term “vaccination” as used herein refers to the introduction of vaccine into a body of a mammalian subject.

The present invention is further illustrated by the following examples, which are provided for the purpose of demonstration rather than limitation. Those of skill in the art should, in light of the present disclosure, appreciate that many changes can be made in the specific embodiments which are disclosed and still obtain a like or similar result without departing from the spirit and scope of the invention.

### EXAMPLES

### Example 1

### Preparation of Immunogenic Compositions Against SARS-CoV-2

Construct of S-2P recombinant protein derived from SARS-COV-2 WT strain (S-2P W recombinant protein). A plasmid having a polynucleotide encoding the residues 14-1208 of S protein of SARS-COV-2 WT strain (wild type, GenBank accession number: MN908947) with proline substitutions at residues 986 and 987, a “GSAS” substitution at the furin cleavage site (residues 682-685) (SEQ ID NO: 1) and a C-terminal T4 fibritin trimerization domain (SEQ ID NO: 2), an HRV3C protease cleavage site (SEQ ID NO: 3), an 8x His Tag, and a Twin-Strep Tag (SEQ ID NO: 4) was transfected into ExpiCHO-S cells (Thermo Fisher Scientific, Waltham, MA, USA).

Construct of S-2P recombinant protein derived from SARS-COV-2 Beta variant (S-2P Beta recombinant protein). The amino acid sequence of S protein of SARS-COV-2 Beta variant has the following substitutions compared to the S protein of SARS-COV-2 WT strain (wild type): D80A, D215G, 241del, 242del, 243del, K417N, E484K, N501Y, D614G, A701V (https://www.cdc.gov/coronavirus/2019-ncov/variants/variant-info.html). (https://www.acep.org/corona/covid-19-field-guide/characteristics-of-covid-19-variants-and-mutants/characteristics-of-covid-19-variants-and-mutants/). A plasmid having a polynucleotide encoding the residues 14-1205 of the S protein of SARS-COV-2 Beta variant with proline substitutions at residues 983 and 984, a “GSAS” substitution at the furin cleavage site (residues 679-682) (SEQ ID NO: 13) and a C-terminal T4 fibritin trimerization domain (SEQ ID NO: 2), an HRV3C protease cleavage site (SEQ ID NO: 3), an 8x His Tag, and a Twin-Strep Tag (SEQ ID NO: 4) was transfected into ExpiCHO-S cells (Thermo Fisher Scientific, Waltham, MA, USA).

Production of S-2P recombinant proteins and formulation of COVID-19 vaccines. Cell culture was harvested after 6 days, and protein was purified from the supernatant using Strep-Tactin resin (IBA Lifesciences, Göttingen, Germany). HRV3C protease (1% wt/wt) was added to the protein and the reaction was incubated overnight at 4° C. The digested protein was further purified using a Superose 6 16/70 column (GE Healthcare Biosciences, Chicago, IL, USA). The purified S-2P W recombinant protein (SEQ ID NO: 5 or 6) or the purified S-2P Beta recombinant protein (SEQ ID NO: 14 or 15) was then formulated with an unmethylated CpG motif (CpG 1018 adjuvant, SEQ ID NO: 8) and/or aluminum-containing adjuvant, such as aluminum hydroxide (Al(OH)3) as the immunogenic compositions against SARS-COV-2.

### Example 2

### Protection from SARS-COV-2 Delta Variant Challenge by the Immunogenic Compositions Containing S-2P W Recombinant Protein or S-2P Beta Recombinant Protein in Hamster

This example provides a description of preclinical studies to assess the immunogenicity of the immunogenic compositions obtained from Example 1 against different SARS-COV-2 strains in hamster.

### Materials and Methods

Immunization and challenge of hamsters. Female golden Syrian hamsters aged 8-10 weeks at study initiation were obtained from the Laboratory Animal Center (Taipei, Taiwan). The hamsters were randomized from different litters into 4 groups (n=10 for each group). Study design of this Example is shown in Table 1. Hamsters in Group 1 were vaccinated on days 22 and 43 with I μg of S-2P W recombinant protein along with 150 μg CpG 1018 adjuvant and 75 μg aluminum hydroxide (alum) (Group 1, W+W). Hamsters in Group 2 were vaccinated on days 1, 22 and 43 with 1 μg of S-2P W recombinant protein along with 150 μg CpG 1018 adjuvant and 75 μg alum (Group 2, W+W+W). Hamsters in Group 3 were vaccinated on days 1 and 22 with 1 μg of S-2P W recombinant protein along with 150 μg CpG 1018 adjuvant and 75 μg alum, and on day 43 with 1 μg of S-2P Beta recombinant protein along with 150 μg CpG 1018 adjuvant and 75 μg alum (Group 3, W+W+B). Hamsters in Group 4 served as an adjuvant control and were vaccinated with only 150 μg CpG 1018 adjuvant and 75 μg alum on days 1, 22, and 43. All hamsters were vaccinated intramuscularly. Serum samples were collected on day 36 (for Groups 2, 3, and 4), day 57 (for all groups), and day 78 (for all groups) via cardiac puncture to confirm presence of neutralizing antibodies. The immunogenicity of the vaccines was determined by neutralization assay with SARS-COV-2 live virus (wild-type and the Alpha, Beta, Gamma, and Delta variants) and SARS-COV-2 pseudovirus (wild-type and the Alpha, Beta, Delta, Lambda, and Mu variants). Hamsters were challenged on day 96 with 1×10⁴ PFU of SARS-COV-2 TCDC#1144 (Strain B.1.617.2, Delta variant, GISAID accession ID: EPI_ISL_2029113) intranasally in a volume of 100 μL per hamster. Then, the hamsters were divided into two cohorts to be euthanized on day 99 and 102 for analyses of viral RNA in lung, infectious viral load (TCID₅₀) in lung, and lung histopathology. The right lung was collected for viral RNA and infectious viral load determination. The left lung was fixed in 4% paraformaldehyde for histopathological examination.

TABLE 1
Study design of hamster challenge study

| | | | Days | | | | | | | | |
|---|---|---|---|---|---|---|---|---|---|---|---|
| Group | S-2P | Adjuvant | 1 | 22 | 36 | 43 | 57 | 78 | 96 | 99 | 102 |
| 1 | W + W | CpG/Alum | | IM | | IM | bleed | bleed | Chall. | Sac. | Sac. |
| | | | | | | | | | | n = 5 | n = 5 |
| 2 | W + W + W | CpG/Alum | IM | IM | bleed | IM | bleed | bleed | Chall. | Sac. | Sac. |
| | | | | | | | | | | n = 5 | n = 5 |
| 3 | W + W + B | CpG/Alum | IM | IM | bleed | IM | bleed | bleed | Chall. | Sac. | Sac. |
| | | | | | | | | | | n = 5 | n = 5 |
| 4 | None | CpG/Alum | IM | IM | bleed | IM | bleed | bleed | Chall. | Sac. | Sac. |
| | | | | | | | | | | n = 5 | n = 5 |

W: S-2P recombinant protein derived from SARS-CoV-2 WT strain (wild type)
B: S-2P recombinant protein derived from SARS-CoV-2 Beta variant
IM: intramuscular injection
Chall.: challenging with SARS-CoV-2 Delta variant
Sac.: sacrifice

SARS-COV-2 Live Virus Neutralization Assay. Different strains of SARS-COV-2 virus, WT (wild type), B.1.1.7 (Alpha), B.1.351 (Beta), P.1 (Gamma), and B.1.617.2 (Delta), were titrated to obtain TCID₅₀. Vero E6 cells (2.5×10⁴ cells/well) were seeded in 96-well plates and incubated. The sera underwent two-fold dilutions with the final dilution being 1:25,600, and the diluted sera were mixed with equal volume of viral solution containing 100 TCID₅₀. The serum-virus mixture was incubated and then added to the plates containing the Vero E6 cells, followed by further incubation. The neutralizing titer was defined as the reciprocal of the highest dilution capable of inhibiting 50% of cytopathic effect (CPE NT₅₀), which was calculated in using the Reed-Muench method.

Pseudovirus production and titration. To produce SARS-COV-2 pseudovirus, a plasmid expressing full-length SARS-COV-2 spike protein was co-transfected into HEK293T cells with packaging and reporter plasmids pCMVA8.91 and pLAS2w.FLuc.Ppuro (RNAi Core, Academia Sinica), using TransIT-LTI transfection reagent (Mirus Bio). The plasmid expresses the full-length SARS-COV-2 spike protein of one of the following SARS-COV-2 strains/variants: WT strain (wild-type; GenBank Accession No. MN908947), B.1.1.7 (Alpha variant, having the following substitutions compared to the S protein of the wild-type: 69del, 70del, 144del, (E484K*), (S494P*), N501Y, A570D, D614G, P681H, T716I, S982A, D1118H (K1191N*)), B.1.351 (Beta variant, having the following substitutions compared to the S protein of the wild-type: D80A, D215G, 241del, 242del, 243del, K417N, E484K, N501Y, D614G, A701V), B.1.617.2 (Delta variant, having the following substitutions compared to the S protein of the wild-type: T19R, G142D, 156-157del, R158G, L452R, T478K, D614G, P681R, D950N), AY.1 (Delta variant, having the following substitutions compared to the S protein of the wild-type: T19R, T95I, G142D, 156-157del, R158G, W258L, K417N, L452R, T478K, D614G, P681R, D950N), C.37 (Lambda variant, having the following substitutions compared to the S protein of the wild-type: G75V, T76I, R246N, 247-253del, L452Q, F490S, D614G, T859N), and B.621 (Mu variant, having the following substitutions compared to the S protein of the wild-type: T951, Y144S, Y145N, R346K, E484K, N501Y, D614G, P681H, D950N). Mock pseudoviruses were produced by omitting the p2019-nCOV spike (WT). Seventy-two (72) hours post-transfection, supernatants were collected, filtered, and frozen at −80 ° C. The transduction unit (TU) of SARS-COV-2 pseudotyped lentivirus was estimated by using cell viability assay in response to the limited dilution of lentivirus. In brief, HEK-293 T cells stably expressing human ACE2 gene were plated on 96-well plate 1 day before lentivirus transduction. For the titration of pseudovirus, different amounts of pseudovirus were added into the culture medium containing polybrene. Spin infection was carried out at 1100×g in 96-well plate for 30 minutes at 37° C. After incubating cells at 37° C. for 16 hours, the culture media containing virus and polybrene were removed and replaced with fresh complete DMEM containing 2.5 μg/ml puromycin. After treating with puromycin for 48 hours, the culture media were removed and cell viability was detected by using 10% AlarmaBlue reagents according to manufacturer's instruction. The survival rate of uninfected cells (without puromycin treatment) was set as 100%. The virus titer (transduction units) was determined by plotting the survival cells versus diluted viral dose.

Pseudovirus-based neutralization assay. HEK293-hAce2 cells (2×10⁴ cells/well) were seeded in 96-well white isoplates and incubated for overnight. Sera were heated at 56° C. for 30 minutes to inactivate complement and diluted in MEM supplemented with 2% FBS at an initial dilution factor of 100, and then twofold serial dilutions were carried out (for a total of 8 dilution steps to a final dilution of 1:25,600). The diluted sera were mixed with an equal volume of pseudovirus (1000 TU) and incubated at 37° C. for 1 hour before adding to the plates with cells. After the 1-hour incubation, the culture medium was replaced with 50 μL of fresh medium. On the following day, the culture medium was replaced with 100 μL of fresh medium. Cells were lysed at 72 hours post infections and relative luciferase units (RLU) were measured. The luciferase activity was detected by Tecan i-control (Infinite 500). The 50% inhibition dilution titers (ID₅₀) were calculated considering uninfected cells as 100% neutralization and cells transduced with only virus as 0% neutralization. Reciprocal ID₅₀ geometric mean titers (GMT) were determined as ID₅₀ titer.

Quantification of viral titer in lung tissue by cell culture infectious assay (TCID₅₀). The middle, inferior, and post-caval lung lobes of hamsters were homogenized in 600 μl of DMEM with 2% FBS and 1% penicillin/streptomycin using a homogenizer. Tissue homogenate was centrifuged at 15,000 rpm for 5 minutes and the supernatant was collected for live virus titration. Briefly, 10-fold serial dilutions of each sample were added onto Vero E6 cell monolayer in quadruplicate and incubated for 4 days. Cells were then fixed with 10% formaldehyde and stained with 0.5% crystal violet for 20 minutes. The plates were washed with tap water and scored for infection. The fifty-percent tissue culture infectious dose (TCID₅₀)/mL was calculated by the Reed and Muench method (Reed and Muench, American Journal of Epidemiology, 27(3): 493-497, 1938).

Real-time RT-PCR for SARS-COV-2 RNA quantification. To measure the RNA levels of SARS-COV-2 Delta variant, specific primers targeting 26,141 to 26,253 region of the envelope (E) gene of SARS-COV-2 Delta genome were used in a TaqMan real-time RT-PCR method (Corman et al., Eurosurveillance. 25(3): 2000045, 2020). Forward primer E-Sarbeco-F1 5′-ACAGGTACGTTAATAGTTAATAGCGT-3′ (SEQ ID NO: 15) and the reverse primer E-Sarbeco-R2 5′-ATATTGCAGCAGTACGCACACA-3′ (SEQ ID NO: 16), in addition to the probe E-Sarbeco-P1 5′-FAM-ACACTAGCCATCCTTACTGCGCTTCG-BBQ-3′ (SEQ ID NO: 17) were used. A total of 30 μL RNA solution was collected from each lung sample using RNeasy Mini Kit (QIAGEN, Germany) according to the manufacturer's instructions. Five (5) μL of RNA sample was added into a total 25 μL mixture of the Superscript III one-step RT-PCR system with Platinum Taq Polymerase (Thermo Fisher Scientific, USA). The final reaction mix contained 400 nM forward and reverse primers, 200 nM probe, 1.6 mM of deoxyribonucleoside triphosphate (dNTP), 4 mM magnesium sulfate, 50 nM ROX reference dye, and 1 μL of enzyme mixture. Cycling conditions were performed using a one-step PCR protocol: 55° C. for 10 minutes for first-strand cDNA synthesis, followed by 3 minutes at 94° C. and 45 amplification cycles at 94° C. for 15 sec and 58° C. for 30 sec. Data was collected and calculated by Applied Biosystems 7500 Real-Time PCR System (Thermo Fisher Scientific, USA). A synthetic 113-bp oligonucleotide fragment was used as a qPCR standard to estimate copy numbers of the viral genome. The oligonucleotides were synthesized by Genomics BioSci and Tech Co. Ltd. (Taipei, Taiwan).

Linear regression analyses. Simple linear regressions were applied to investigate the correlation between viral RNA and the neutralizing titer (NT₅₀) against SARS-COV-2 Delta variant. All analyses were preformed using Prism 6.01 (GraphPad) software.

Histopathology. The left lungs of the tested hamsters were fixed in 4% paraformaldehyde for histopathological examination. After fixation with 4% paraformaldehyde for one week, the lung was trimmed, processed, embedded, sectioned, and stained with Hematoxylin and Eosin (H&E), followed by microscopic examination. The lung section was evaluated with a lung histopathological scoring system. The section was divided into 9 areas. Lung tissue of each area was scored using the following scoring system: “0”—normal, no significant finding; “1”—minor inflammation with a slight thickening of alveolar septa and sparse monocyte infiltration; “2”—apparent inflammation, alveolus septa thickening with more interstitial mononuclear inflammatory infiltration; “3”—diffuse alveolar damage (DAD), with alveolus septa thickening, and increased infiltration of inflammatory cells; “4”—DAD, with extensive exudation and septa thickening, shrinking of alveoli, the restricted fusion of the thick septa, obvious septa hemorrhage, and more cell infiltration in alveolar cavities; “5”—DAD, with massive cell filtration in alveolar cavities and alveoli shrinking, sheets of septa fusion, and hyaline membranes lining the alveolar walls. The average scores of these 9 areas are used to represent the score of the animal.

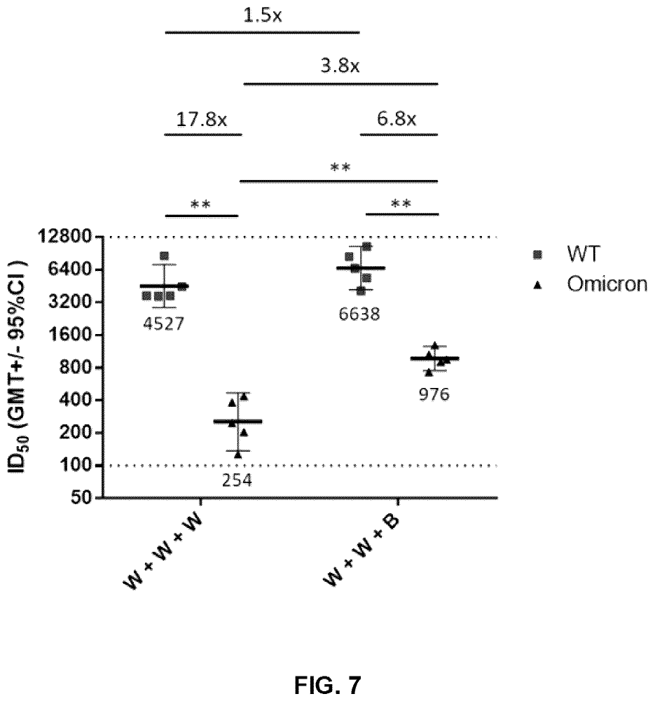

Statistical analysis. Prism 6.01 (GraphPad Software Inc., San Diego, CA, USA) was used for statistical analysis. Mann-Whitney test, Kruskal-Wallis with corrected Dunn's multiple comparisons test, and two-way ANOVA with Dunnett test for multiple comparison were used to calculate significance where appropriate. Spearman's rank correlation coefficient and linear regression were calculated for FIG. 7. *p<0.05, **p<0.01, ***p<0.001, ****p<0.0001.

### Results

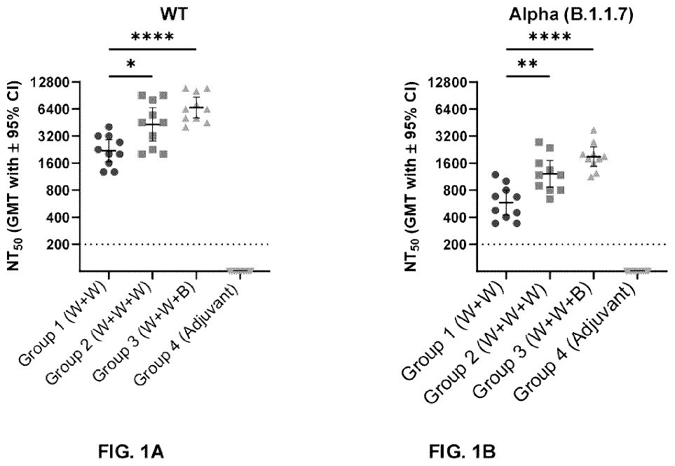

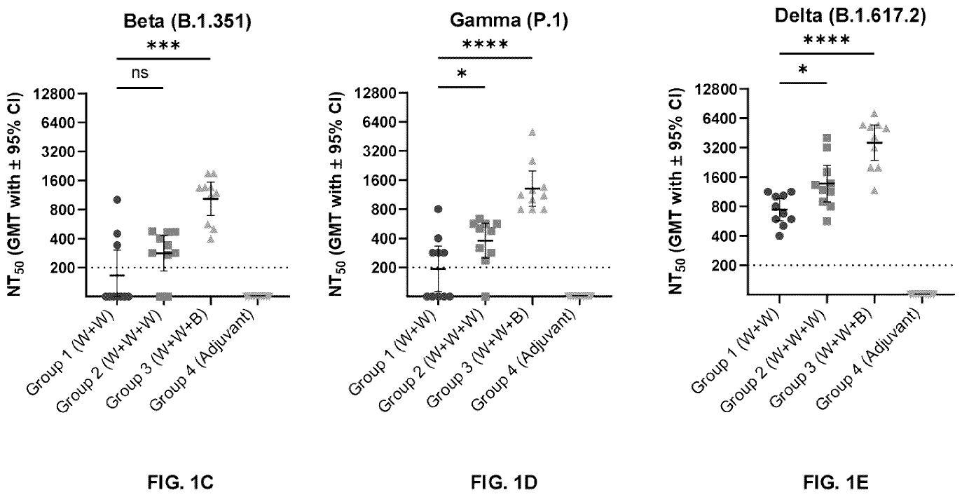

Administration of two doses of S-2P W recombinant protein followed by one dose of S-2P Beta recombinant protein to hamsters induced higher level of neutralizing antibodies against SARS-COV-2, including wild-type and different variants, than administration of two or three doses of S-2P W recombinant protein. Hamsters were divided into 4 groups receiving two or three doses adjuvanted S-2P recombinant proteins (S-2P W and/or S-2P Beta) at 21 days apart. No mortality, abnormality of clinical signs, differences in body weight changes, nor food consumption were observed after vaccination among the 4 groups, indicating multiple doses of S-2P recombinant proteins (either S-2P W or S-2P Beta) do not result in systemic adverse effects in hamsters. Five weeks (day 78) after the last (either second or third) immunization, highest levels of neutralizing antibody titers against live viruses of SARS-COV-2 wild type (FIG. 1A), B.1.1.7 (Alpha, FIG. 1B), B.1.351 (Beta, FIG. 1C), P.1 (Gamma, FIG. 1D), and B.1.617.2 (Delta, FIG. 1E) variants were all found in the group receiving two doses of S-2P W recombinant protein followed by one dose of S-2P Beta recombinant protein (Group 3, W+W+B). The result suggests that the vaccine combination of two doses of S-2P W recombinant protein followed by one dose of S-2P Beta recombinant protein generates better protection rate against SARS-COV-2 live virus, including wild type and the Alpha, Beta, Gamma, and Delta variants, than the vaccine combinations of two or three doses of S-2P W recombinant protein.

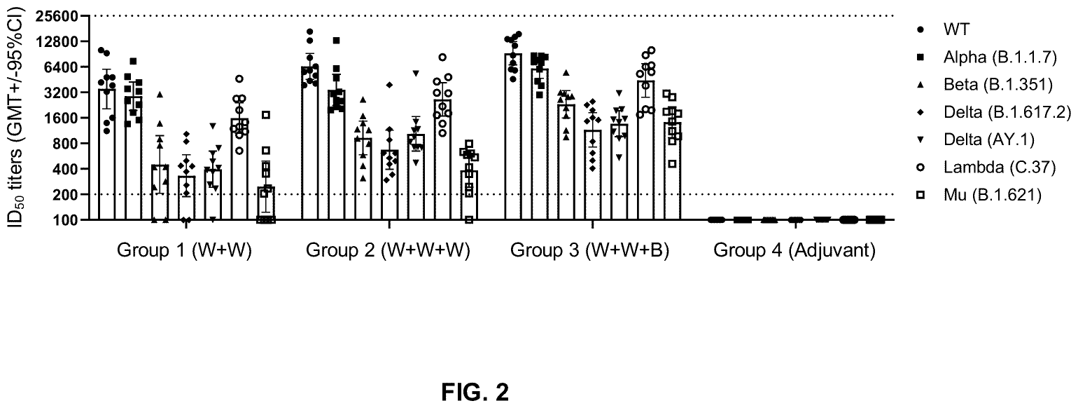

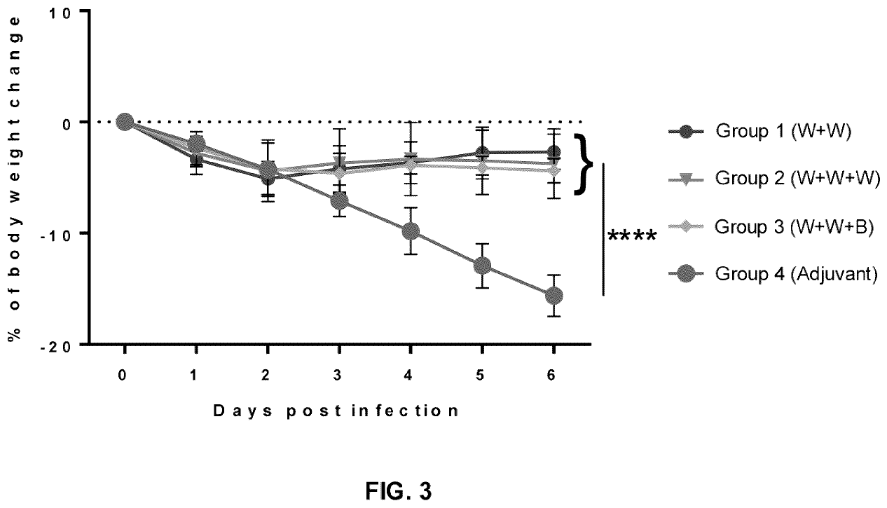

Five (5) weeks (day 78) after the last (either second or third) immunization, antisera were also collected for pseudovirus-based neutralization assays. Again, highest levels of neutralizing antibody titers against pseudoviruses of SARS-COV-2 wild type and B.1.1.7 (Alpha), B.1.351 (Beta), B.1.617.2 (Delta), AY.1 (Delta), C.37 (Lambda), and B.1.621 (Mu) variants were all found in the group receiving two doses of S-2P W recombinant protein followed by one dose of S-2P Beta recombinant protein (Group 3, W+W+B) (FIG. 2). Hamsters receiving at least two doses of S-2P W recombinant protein with or without a third booster (i.e., Groups 1-3) all produced high level of neutralizing antibody against pseudoviruses of SARS-COV-2 wild type and B.1.1.7 (Alpha) and C.37 (Lambda) variants (FIG. 3). However, only hamsters receiving two doses of S-2P W recombinant protein followed by one dose of S-2P Beta recombinant protein (Group 3, W+W+B) produced high level of neutralizing antibody against pseudoviruses of SARS-CoV-2 B.1.351 (Beta), B.1.617.2 (Delta), AY.1 (Delta), and B.1.621 (Mu) variants (FIG. 2). The results of pseudovirus-based neutralization assays indicate that administration of two doses of S-2P W recombinant protein followed by one dose of S-2P Beta recombinant protein (Group 3, W+W+B) provides a broader spectrum of protection against wild-type SARS-COV-2 and different variants, including B.1.1.7 (Alpha), B.1.351 (Beta), B.1.617.2 (Delta), AY.1 (Delta), C.37 (Lambda), and B.1.621 (Mu) variants, than administration of two or three doses of S-2P W recombinant protein (Group 1 or 2, W+W or W+W+W).

Vaccines containing S-2P recombinant protein protect hamsters from clinical signs and reduce infectious viral load in hamsters after challenged with SARS-COV-2 Delta variant. Fifty-three (53) days after completion of immunization (Day 96), hamsters were challenged with 10⁴ PFU of the Delta variant and body weights were tracked up to 6 days post infection (d.p.i.). All the vaccinated groups (Groups 1 to 3) did not show weight loss up to 6 days after virus challenge, compared with the adjuvant control (Group 4).

The protective effect was most significant at 6 d.p.i. in vaccinated groups, while the adjuvant only group experienced significant weight loss (FIG. 3). The result suggests that hamsters receiving vaccines containing S-2P W and/or Beta recombinant protein (either two doses or three doses) were protected from clinical signs after challenged with SARS-CoV-2 Delta variant.

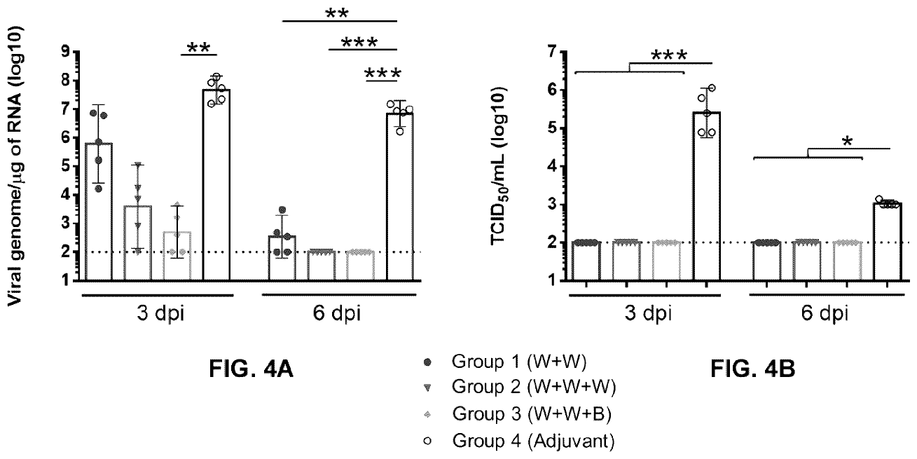

Lung viral RNA in hamsters from the three vaccination groups were lower than that of the adjuvant control; however, only hamsters administered with two doses of S-2P W recombinant protein followed by one dose of S-2P Beta recombinant protein (Group 3) had significantly lower level of viral RNA in their lungs than the adjuvant control at 3 dpi (FIG. 4A). The results indicate that administering 3 doses of S-2P recombinant protein provides enough protection against SARS-COV-2 Delta variant, especially administering two doses of S-2P W recombinant protein followed by one dose of S-2P Beta recombinant protein.

In addition, infectious viral load (TCID₅₀) cannot be detected in lungs of hamsters vaccinated with either two or three doses of S-2P recombinant protein (Groups 1 to 3) at 3 and 6 dpi (FIG. 4B). Note that infectious viral load dropped noticeably at 6 dpi in adjuvant control group (Group 4) due to hamsters' natural immune response. The differences between the detection of lung viral RNA in the vaccination groups (Groups 1 to 3) at 3 dpi and the undetectable infectious viral load (TCID₅₀) in lung in Groups 1 to 3 at 3 dpi may be caused by the different nature of the two test methods. Real-time RT-PCR detects fragments of viral RNA from both live and dead virus, whereas cell culture infectious assay (TCID₅₀) detects only live replicating virus. The results suggest that administering at least two doses of S-2P W recombinant protein protects the subjects from infection of SARS-COV-2 Delta variant.

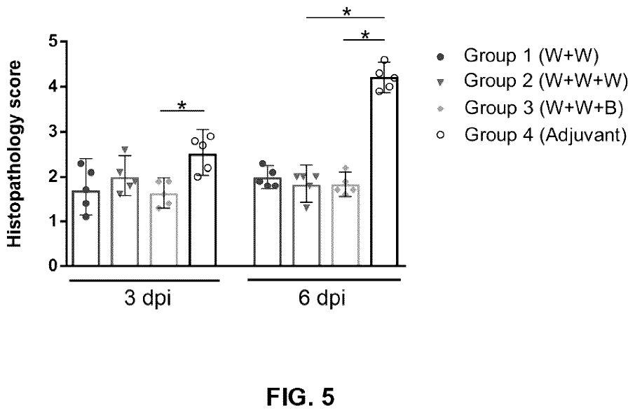

Lung sections were analyzed, and pathology scoring was tabulated (FIG. 5). There were no differences at 3 dpi between control and experimental groups; however, at 6 dpi, the adjuvant control group (Group 4) had significantly increased lung pathology including extensively severe immune cell infiltration, hemorrhage, and diffuse alveolar damage, compared to groups receiving three doses of S-2P recombinant protein (Groups 2 and 3) (FIG. 5). These results showed that administering two doses of S-2P W recombinant protein followed by an extra boost dose of S-2P recombinant protein (either S-2P W or S-2P Beta) induced a robust immune response to protect hamsters from the infection of SARS-COV-2 Delta variant, including being able to suppress viral load in lungs and prevent weight loss and lung pathology in infected hamsters.

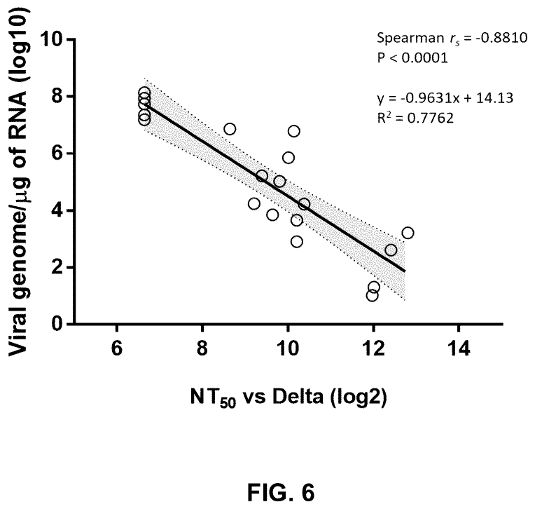

Viral RNA is negatively and significantly associated with the neutralizing titer (NT₅₀) against SARS-COV-2 Delta variant. To estimate the relationships between viral RNA and the neutralizing titer (NT₅₀) against SARS-COV-2 Delta variant, linear regressions were applied to the dataset of the hamster challenge study. Viral RNA is negatively associated with the neutralizing titer (NT₅₀) against SARS-COV-2 Delta variant (Spearman rₛ=−0.8810, R²=0.7762, p<0.0001) (FIG. 6). The results suggest that the level of neutralizing titer (NT₅₀) after immunization could be predictive of clearance of viral RNA in the lungs post challenge.

In conclusion, the hamster study shows that administration of two doses of S-2P W recombinant protein followed by one dose of S-2P Beta recombinant protein induces a higher level of neutralizing antibodies against SARS-COV-2, including wild type strain and various variants, and provide a broader spectrum of protection against SARS-COV-2 variants than administration of two or three doses of S-2P W recombinant protein. Therefore, the dosing regimen serves as a good strategy to increase immunity against SARS-COV-2 variants, especially Variants of Concern (VoCs).

### Example 3

### Evaluation of the Neutralizing Ability of the Immunogenic Compositions Containing S-2P W Recombinant Protein or S-2P Beta Recombinant Protein Against SARS-COV-2 Omicron Variant in Hamster

This example provides a description of preclinical studies to assess the immunogenicity of the immunogenic compositions obtained from Example 1 against the Omicron variant of SARS-COV-2 in hamster.

### Materials and Methods

Immunization of hamsters. Female golden Syrian hamsters aged 8-10 weeks at study initiation were obtained from the Laboratory Animal Center (Taipei, Taiwan). The hamsters were randomized from different litters into 2 groups (n=10 for each group). Hamsters in Group 1 were vaccinated on days 1, 22 and 43 with 1 μg of S-2P W recombinant protein along with 150 μg CpG 1018 adjuvant and 75 μg alum (Group 1, W+W+W). Hamsters in Group 2 were vaccinated on days 1 and 22 with 1 μg of S-2P W recombinant protein along with 150 μg CpG 1018 adjuvant and 75 μg alum, and on day 43 with 1 μg of S-2P Beta recombinant protein along with 150 μg CpG 1018 adjuvant and 75 μg alum (Group 2, W+W+B). All hamsters were vaccinated intramuscularly. Serum samples were collected on day 78 via cardiac puncture to confirm presence of neutralizing antibodies. The immunogenicity of the vaccines was determined by neutralization assay with SARS-COV-2 Omicron variant pseudovirus.

Pseudovirus production and titration. The production and titration of SARS-CoV-2 WT strain (wild type) and Omicron variant (B.1.1.529 or BA.1, having the following substitutions compared to the S protein of the wild-type: A67V, 469-70, T951, G142D, A143-145, 4211, L212I, ins214EPE, G339D, S371L, S373P, S375F, K417N, N440K, G446S, S477N, T478K, E484A, Q493R, G496S, Q498R, N501Y, Y505H, T547K, D614G, H655Y, N679K, P681H, N764K, D796Y, N856K, Q954H, N969K, and L981F) pseudovirus are the same as the description in Example 2.

Pseudovirus-based neutralization assay. The neutralization assay based on SARS-COV-2 WT strain (wild type) and Omicron variant (B.1.1.529 or BA.1) pseudovirus is the same as the description in Example 2.

Statistical analysis. Prism 6.01 (GraphPad Software Inc., San Diego, CA, USA) was used for statistical analysis. Mann-Whitney test was used to calculate significance. **p<0.01.

### Results

Administration of two doses of S-2P W recombinant protein followed by one dose of S-2P Beta recombinant protein to hamsters induced a higher level of neutralizing antibodies against SARS-COV-2 Omicron variant than administration of three doses of S-2P W recombinant protein. Hamsters were divided into 2 groups receiving three doses adjuvanted S-2P recombinant proteins (3 doses of S-2P W or 2 doses of S-2P W followed by 1 dose of S-2P Beta) at 21 days apart. No mortality, abnormality of clinical signs, differences in body weight changes, nor food consumption were observed after vaccination among the 2 groups, indicating multiple doses of S-2P recombinant proteins (either S-2P W or S-2P Beta) do not result in systemic adverse effects in hamsters. Five (5) weeks (day 78) after the third immunization, antisera were collected for pseudovirus-based neutralization assays. Compared to hamsters receiving three doses of S-2P W recombinant protein (Group 1, W+W+W), hamsters receiving two doses of S-2P W recombinant protein followed by one dose of S-2P Beta recombinant protein (Group 2, W+W+B) have higher levels of neutralizing antibody against pseudoviruses of SARS-CoV-2 wild type and B.1.1.529 (Omicron), especially a significantly higher level of neutralizing antibody against pseudoviruses of SARS-COV-2 B.1.1.529 (Omicron) (FIG. 7).

The results of pseudovirus-based neutralization assays suggest that administration of two doses of S-2P W recombinant protein followed by one dose of S-2P Beta recombinant protein (Group 2, W+W+B) provides better protection against wild-type SARS-COV-2 and the B.1.1.529 (Omicron) variant than administration of three doses of S-2P W recombinant protein (Group 1, W+W+W).

### Example 4

### Evaluation of Thermostability of the Immunogenic Composition Containing S-2P Beta Recombinant Protein Stored at 40° C. to 42° C.

This example provides a description of studies to assess thermostability of the immunogenic composition containing S-2P Beta recombinant protein obtained from Example 1.

### Materials and Methods

The immunogenic composition containing the S-2P Beta recombinant protein obtained from Example 1 was stored at 40° C. for 3 days, 40° C. for 7 days, 42° C. for 3 days, or 42° C. for 7 days for the thermostability test. The same immunogenic composition constantly stored at 4° C. was used as control.

Mouse immunizations. BALB/c mice aged 6-8 weeks (N =5/group) were randomized from different litters into 5 groups (n=5 for each group). Mice were vaccinated on days 1 and 22 (i.e., 3 weeks apart) with 3 μg of S-2P Beta recombinant protein along with 150 μg CpG 1018 adjuvant and 75 μg aluminum hydroxide (alum) stored at 40° C. for 3 days (Group 1), stored at 40° C. for 7 days (Group 2), stored at 42° C. for 3 days (Group 3), stored at 42° C. for 7 days (Group 4), or constantly stored at 4° C. (Group 5, served as control). All mice were vaccinated intramuscularly. Two weeks after the final immunization, sera were collected for measurement of antibody responses.

Pseudovirus production and titration. The production and titration of SARS-CoV-2 Beta variant (B.1.351) pseudovirus are the same as the description in Example 2.

Pseudovirus-based neutralization assay. The neutralization assay based on SARS-COV-2 Beta variant (B.1.351) pseudovirus is the same as the description in Example 2.

Statistical analysis. Prism 6.01 (GraphPad Software Inc., San Diego, CA, USA) was used for statistical analysis. Mann-Whitney test was used to calculate significance.

### Results

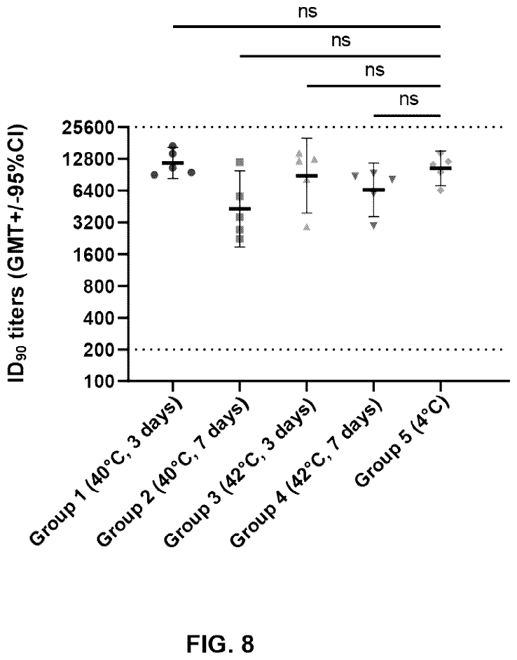

The immunogenic composition of the present invention remains stable at up to 42° C. for 7 days. Mice were divided into 5 groups receiving two doses adjuvanted S-2P Beta recombinant protein stored at 40-42° C. for 3 to 7 days at 21 days apart. No mortality, abnormality of clinical signs, differences in body weight changes, body temperature, nor food consumption were observed after vaccination among the 5 groups, indicating multiple doses of S-2P Beta recombinant proteins stored at 40-42° C. for 3 to 7 days do not result in systemic adverse effects in mice. Fourteen (14) days after the second immunization, neutralizing antibody titers were analyzed, and the results are shown in FIG. 8.

Mice administered with two doses of S-2P Beta recombinant protein stored at 40° C. for 3 days (Group 1), stored at 40° C. for 7 days (Group 2), stored at 42° C. for 3 days (Group 3), stored at 42° C. for 7 days (Group 4), and constantly stored at 4° C. (Group 5, control group) have similar level of neutralization antibodies against SARS-COV-2 Beta variant. There is no statistically significant difference between the immunogenic composition constantly stored at 4° C. (Group 5, control group) and the same immunogenic composition stored at 40° C. for 3 days (Group 1), stored at 40° C. for 7 days (Group 2), stored at 42° C. for 3 days (Group 3), or stored at 42° C. for 7 days (Group 4) (FIG. 8). The results indicate that the immunogenic composition of the present invention remains stable under the condition of high temperature (at least)42° C. for at least a period of time (at least 7 days).

### Example 5

### Evaluation of the Neutralizing Ability of the Immunogenic Compositions Containing S-2P W Recombinant Protein or S-2P Beta Recombinant Protein Against SARS-COV-2 Variants in Mice

This example provides a description of preclinical studies to assess the immunogenicity of the immunogenic compositions obtained from Example 1 against different SARS-COV-2 strains in mice.

### Materials and Methods

Mouse immunizations. BALB/c mice aged 6-8 weeks (N=5/group) were randomized from different litters into 7 groups (n=5 for each group). Mice in Group 1 were vaccinated on days 1 and 22 (i.e., 3 weeks apart) with 3 μg of S-2P W recombinant protein along with 150 μg CpG 1018 adjuvant and 75 μg alum (Group 1, W+W). Mice in Group 2 were vaccinated on days 1 and 22 (i.e., 3 weeks apart) with 3 μg of S-2P Beta recombinant protein along with 150 μg CpG 1018 adjuvant and 75 μg alum (Group 2, B+B). Mice in Group 3 were vaccinated on day 1 with 3 μg of S-2P W recombinant protein along with 150 μg CpG 1018 adjuvant and 75 μg alum, and on day 22 (i.e., 3 weeks apart) with 3 μg of S-2P Beta recombinant protein along with 150 μg CpG 1018 adjuvant and 75 μg alum (Group 3, W+B). Mice in Group 4 were vaccinated on days 1, 22, and 43 (i.e., 3 weeks apart) with 3 μg of S-2P W recombinant protein along with 150 μg CpG 1018 adjuvant and 75 μg alum (Group 4, W+W+W). Mice in Group 5 were vaccinated on days 1 and 22 (i.e., 3 weeks apart) with 3 μg of S-2P W recombinant protein along with 150 μg CpG 1018 adjuvant and 75 μg alum, and on day 43 (i.e., 3 weeks apart from second shot) with 3 μg of S-2P Beta recombinant protein along with 150 μg CpG 1018 adjuvant and 75 μg alum (Group 5, W+W+B). Mice in Group 6 were vaccinated on days 1, 22, and 43 (i.e., 3 weeks apart) with 3 μg of S-2P Beta recombinant protein along with 150 μg CpG 1018 adjuvant and 75 μg alum (Group 6, B+B+B). Mice in Group 7 served as an adjuvant control and were vaccinated with only 150 μg CpG 1018 adjuvant and 75 μg alum on days 1 and 22 (Group 7, Adjuvant). All mice were vaccinated intramuscularly. Two weeks after the final immunization, sera were collected for measurement of antibody responses.

Pseudovirus production and titration. The production and titration of SARS-CoV-2 WT strain, Beta variant (B.1.351), Delta variant (B.1.617.2), and Omicron variant (B.1.1.529 or BA.1) pseudovirus are the same as the description in Examples 2 and 3.

Pseudovirus-based neutralization assay. The neutralization assay based on SARS-COV-2 WT strain (wild type), Beta variant (B.1.351), Delta variant (B.1.617.2), and Omicron variant (B.1.1.529 or BA.1) pseudovirus is the same as the description in Examples 2 and 3.

Statistical analysis. Prism 6.01 (GraphPad Software Inc., San Diego, CA, USA) was used for statistical analysis. Mann-Whitney test was used to calculate significance. *p<0.05, **p<0.01.

### Results

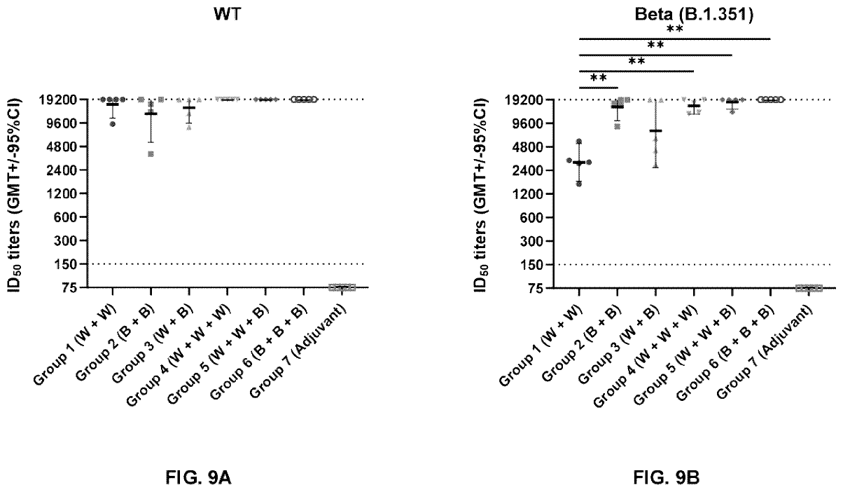

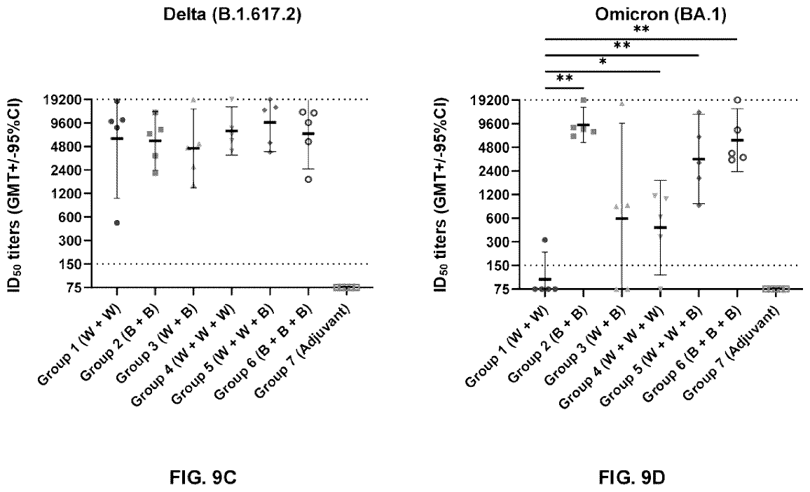

Administration of at least one dose of the Beta recombinant protein in a multiple-dose regimen induced high level of neutralizing antibodies against SARS-COV-2 WT strain, Beta, Delta, and Omicron variants. Mice were divided into 7 groups receiving two or three doses of adjuvanted S-2P recombinant proteins (S-2P W and/or S-2P Beta) at 21 days apart. No mortality, abnormality of clinical signs, differences in body weight changes, body temperature, nor food consumption were observed after vaccination among the 7 groups, indicating multiple doses of S-2P recombinant proteins (either S-2P W or S-2P Beta) do not result in systemic adverse effects in mice. Fourteen (14) days after the last immunization, neutralizing antibody titers were analyzed, and the results are shown in FIGS. 9A to 9D.

Administration of two or three doses of adjuvanted S-2P recombinant proteins (S-2P W and/or S-2P Beta) (Groups 1 to 6) induces high degree of neutralizing antibodies against SARS-COV-2 WT strain (wild type) (FIG. 9A) and the Delta variant (FIG. 9C) in mice. In addition, the combinations of two doses of S-2P Beta recombinant protein (Group 2, B+B), three doses of S-2P W recombinant protein (Group 4, W+W+W), two doses of S-2P W recombinant protein followed by one dose of S-2P Beta recombinant protein (Group 5, W+W+B), and three doses of S-2P Beta recombinant protein (Group 6, B+B+B) induce higher degree of neutralizing antibodies against SARS-COV-2 Beta variant (FIG. 9B) and Omicron variant (FIG. 9D) than the combination of two doses of S-2P W recombinant protein (Group 1, W+W).

The results demonstrate that administration of at least one dose of the Beta recombinant protein in a multiple-dose regimen, such as two or three doses of S-2P Beta recombinant protein, or two doses of S-2P W recombinant protein followed by one dose of S-2P Beta recombinant protein, induces high level of neutralizing antibodies against SARS-COV-2, including wild type strain and various variants, and provide a broad spectrum of protection against SARS-COV-2 variants.

### Example 6

### Safety and Immunogenicity of the Immunogenic Compositions Containing S-2P W Recombinant Protein or S-2P Beta Recombinant Protein in Humans

This Example provides a Phase I study conducted in healthy human subjects to assess safety and immunogenicity of a booster dose of a SARS-COV-2 vaccine containing S-2P W recombinant protein (which is referred to herein as “MVC-COV1901” or “W”) or a SARS-COV-2 vaccine containing S-2P Beta recombinant protein (which is referred to herein as “MVC-COV1901-Beta” or “B”, i.c., the immunogenic composition of the present invention) following 2 or 3 doses of MVC-COV1901. The MVC-COV1901 and MVC-COV1901-Beta SARS-COV-2 vaccines are described in greater detail in Example 1. Vaccines. Each MVC-COV1901 SARS-COV-2 vaccine (W) contains 15 μg of

S-2P W recombinant protein adjuvanted with 750 μg of CpG 1018 adjuvant and 375 μg (Al equivalent to weight) of aluminum hydroxide, administered as a single 0.5 mL intramuscular (IM) injection. Each MVC-COV1901-Beta SARS-COV-2 vaccine (B) contains 15 μg or 25 μg of S-2P Beta recombinant protein adjuvanted with 750 μg of CpG 1018 adjuvant and 375 μg (Al equivalent to weight) of aluminum hydroxide, administered as a single 0.5 mL intramuscular (IM) injection.

Participant. The study enrolled 93 eligible, healthy adults aged between 18 (≥18) and 54 (<55) years. Eligible participants vaccinated with two and three prior doses of MVC-COV1901 were assigned into Group A and B, respectively. Eligibility was determined based on medical history, physical examination, laboratory tests, and investigators' clinical judgment. Exclusion criteria included a history of known potential exposure to SARS COV-1 or 2 viruses, having received any other COVID-19 vaccine, impaired immune function, history of autoimmune disease, uncontrolled HIV, HBV, or HCV infection, abnormal autoantibody tests, febrile or acute illness within 2 days of the booster dose, and acute respiratory illness within 14 days of the booster dose.

Study Design. This study is a prospective, randomized, open-labeled Phase I study to evaluate safety and immunogenicity of a booster dose of MVC-COV1901 (W) (15 μg per dose) or MVC-COV1901-Beta (B) (15 μg or 25 μg per dose) following 2 doses of MVC-COV1901 (Group A) or 3 doses of MVC-COV1901 (Group B). Participants were divided into 6 subgroups (Subgroups A-1, A-2, A-3, B-1, B-2, and B-3). The booster dose was administered by intramuscular (IM) injection in the deltoid muscle of the non-dominant arm. The mean intervals between the last prior dose of MVC-COV1901 and the booster dose were 223.3 to 294.5 days in Group A and 120.9 to 128.0 days in Group B. Group A: Group A includes 38 participants vaccinated with 2 prior doses of

MVC-COV1901 (15 μg per dose) 12 weeks apart. The 38 participants were randomly divided into 3 subgroups:

Subgroup A-1 (W+W+W): Fourteen (14) participants received a booster dose of MVC-COV1901 (15 μg per dose) after 2 prior doses of MVC-COV1901.

Subgroup A-2 (W+W+15B): Twelve (12) participants received a booster dose of MVC-COV1901-Beta (15 μg per dose) after 2 prior doses of MVC-COV1901.

Subgroup A-3 (W+W+25B): Twelve (12) participants received a booster dose of MVC-COV1901-Beta (25 μg per dose) after 2 prior doses of MVC-COV1901.

Group B: Group B includes 55 participants vaccinated with 3 prior doses of MVC-COV1901 (15 μg per dose), in which the first and the second doses were administered 12 weeks apart, and the second and the third doses were administered 12 to 24 weeks apart. The 53 participants were randomly divided into 3 subgroups:

Subgroup B-1 (W+W+W+W): Eighteen (18) participants received a booster dose of MVC-COV1901 (15 μg per dose) after 3 prior doses of MVC-COV1901. Subgroup B-2 (W+W+W+15B): Nineteen (19) participants received a booster dose of MVC-COV1901-Beta (15 μg per dose) after 3 prior doses of MVC-COV1901.

Subgroup B-3 (W+W+W+25B): Eighteen (18) participants received a booster dose of MVC-COV1901-Beta (25 μg per dose) after 3 prior doses of MVC-COV1901.

Vital signs and electrocardiogram (ECG) were performed before and after vaccination. Participants were observed for at least 30 min after the booster dose to identify any immediate adverse events (AEs), and were asked to record solicited local (pain/tenderness, erythema/redness, and induration/swelling) and systemic (fever, malaise/fatigue, myalgia, headache, nausea/vomiting, diarrhea) AEs in the participant's diary card for up to 7 days after the booster dose. Unsolicited AEs were recorded for 28 days following the booster dose; all other AEs, such as serious adverse events (SAEs) and adverse events of special interest (AESIs) were recorded throughout the study period. Serum samples were collected for hematology, biochemistry and immunology evaluation.

The primary immunogenicity endpoints were to evaluate neutralizing antibody against wild type (WT) and Beta variant SARS-COV-2 live virus at Visits 2 (the day of the booster dose; baseline) and 5 (4 weeks after the booster dose), anti-spike immunoglobulin G (IgG) antibody at Visits 2, 4 (2 weeks after the booster dose), and 5, and neutralizing antibody against WT and Omicron variant (BA.4/BA.5 subvariant) pseudovirus at Visits 2 and 4.

SARS-COV-2 Live Virus Neutralization Assay. Wild type SARS-COV-2 virus (hCoV-19/Taiwan/4/2020, GISAID EPI_ISL_411927) and Beta variant (B.1.351, hCoV-19/Taiwan/1013) were used for SARS-COV-2 live virus neutralization assay, which is the same as the description in Example 2.

SARS-COV-2 Spike-Specific IgG. Total serum anti-Spike IgG titers were detected with direct enzyme-linked immunosorbent assay (ELISA) using customized 96-well plates coated with S-2P antigen.

Pseudovirus production and titration. The production and titration of SARS-CoV-2 WT strain (wild type) and Omicron variant (BA.4/BA.5, both possess identical spike protein sequences, having the following substitutions compared to the S protein of the wild-type: T19I, del24-26, A27S, del69-70, G142D, V213G, G339D, S371F, S373P,

S375F, T376A, D405N, R408S, K417N, N440K, L452R, S477N, T478K, E484A, F486V, Q498R, N501Y, Y505H, D614G, H655Y, N679K, P681H, N764K, D796Y, Q954H, and N969K) pseudovirus are the same as the description in Example 2.

Pseudovirus-based neutralization assay. The neutralization assay based on SARS-COV-2 WT strain (wild type) and Omicron variant (BA.4/BA.5) pseudovirus is the same as the description in Example 2.

Statistical Analysis. GMT and corresponding CI are calculated using an ANCOVA model with baseline log-titers, BMI (<30 or ≥30 kg/m²) and comorbidity (yes or no) and sex (male or female) as covariate. Prism 6.01 (GraphPad) was used for statistical analysis. Kruskal-Wallis with corrected Dunn's multiple comparisons test was used for comparison of means of non-parametric dataset.

### Results

Safety. No SAE (grade 3 AEs or higher) or AESI related to the vaccine occurred at this data cut-off point. No study intervention was modified or interrupted.

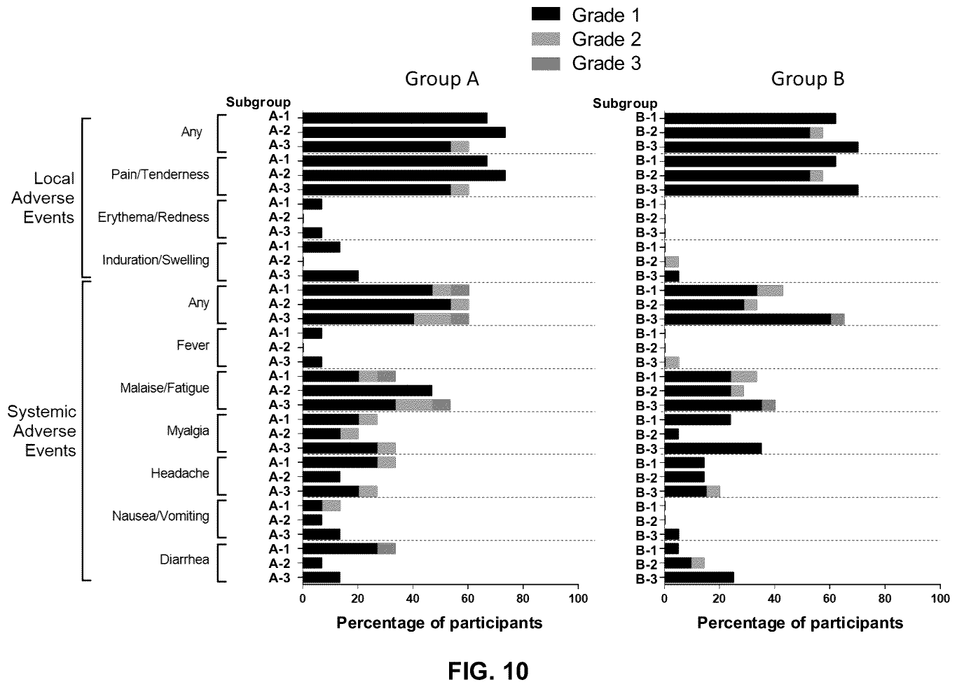

Occurrences of solicited AEs are summarized in FIG. 10. The most commonly reported local AEs were pain/tenderness (60.0˜73.3% in Group A and 57.1˜70.0% in Group B), while malaise/fatigue (33.3˜53.3% in Group A and 28.6˜40.0% in Group B) were the most commonly reported systemic AEs among all treatment groups. Most local and systemic AEs were mild. Evaluation of safety laboratory values, ECG interpretation, and other unsolicited adverse events revealed no specific concern.

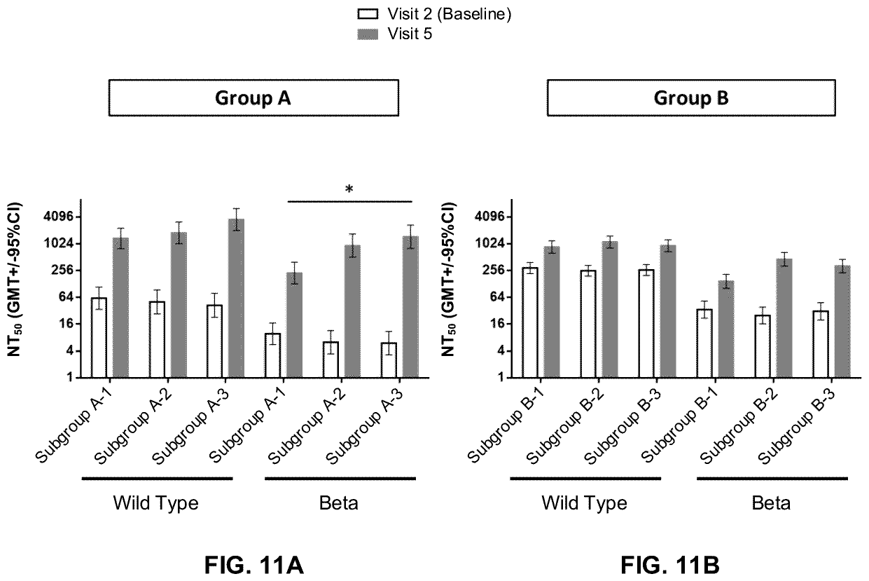

Immunogenicity. The results of live virus neutralizing assay are summarized in FIGS. 11A to 11B. In Group A (FIG. 11A), at Visit 5, a booster dose of 25 μg MVC-COV1901-Beta (Subgroup A-3; W+W+25B) induced the highest levels of neutralizing antibody against wild type (NT₅₀ GMT: 3602.75) and the Beta variant (NT₅₀ GMT: 1476.85) SARS-COV-2. In addition, a booster dose of 15 μg MVC-COV1901-Beta (Subgroup A-2; W+W+15B) induced higher levels of neutralizing antibody against wild type (NT₅₀ GMT: 1805.02) and the Beta variant (NT₅₀ GMT: 931.34) SARS-COV-2 than a booster dose of 15 μg MVC-COV1901 (Subgroup A-1; W+W+W; GMT: 1352.00 and 225.59 for neutralizing antibody against wild type and the Beta variant SARS-COV-2, respectively). In particular, a booster dose of 25 μg MVC-COV1901-Beta (Subgroup A-3; W+W+25B) induced a significantly higher level of neutralizing antibody against the Beta variant SARS-COV-2 virus compared to a booster dose of 15 μg MVC-COV1901 (Subgroup A-1; W+W+W) (p<0.05).

In Group B (FIG. 11B), at Visit 5, a booster dose of 15 μg MVC-COV1901-Beta (Subgroup B-2; W+W+W+15B) induced the highest levels of neutralizing antibody against wild type (NT₅₀ GMT: 1124.98) and the Beta variant (NT₅₀ GMT: 459.23) SARS-COV-2. In addition, a booster dose of 25 μg MVC-COV1901-Beta (Subgroup B-3; W+W+W+25B) induced higher levels of neutralizing antibody against wild type (NT₅₀ GMT: 928.54) and the Beta variant (NT₅₀ GMT:323.78) SARS-COV-2 than a booster dose of 15 μg MVC-COV1901 (Subgroup B-1; W+W+W+W; GMT: 867.93 and 147.14 for neutralizing antibody against wild type and the Beta variant SARS-COV-2, respectively).

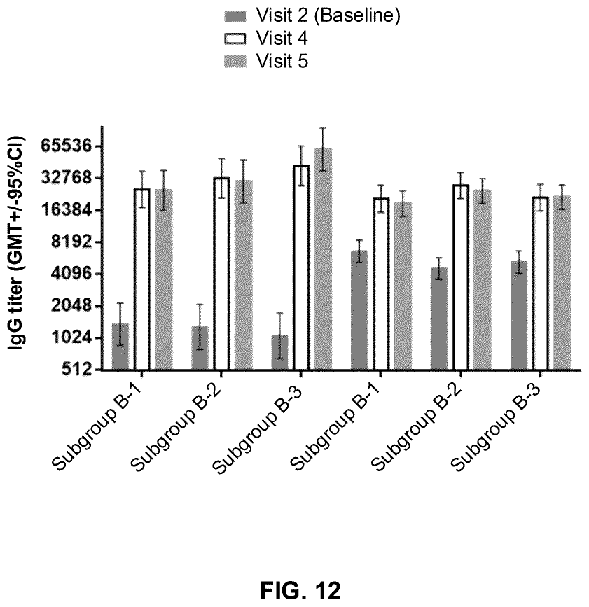

The results of anti-spike IgG titration are summarized in FIG. 12, which are similar to the results of live virus neutralizing assay. In Group A, a booster dose of 25 μg MVC-COV1901-Beta (Subgroup A-3; W+W+25B) induced the highest levels of anti-spike IgG titer (43208.61 at Visit 4 and 61907.00 at Visit 5); in addition, a booster dose of 15 μg MVC-COV1901-Beta (Subgroup A-2; W+W+15B) induced higher levels of anti-spike IgG titer (32938.41 at Visit 4 and 30672.19 at Visit 5) than a booster dose of 15 μg MVC-COV1901 (Subgroup A-1; W+W+W; 25878.60 at Visit 4 and 25257.75 at Visit 5). In Group B, a booster dose of 15 μg MVC-COV1901-Beta (Subgroup B-2; W+W+W+15B) induced the highest levels of anti-spike IgG titer (28179.38 at Visit 4 and 24953.20 at Visit 5); in addition, a booster dose of 25 μg MVC-COV1901-Beta (Subgroup B-3; W+W+W+25B) induced higher levels of anti-spike IgG titer (21638.16 at Visit 4 and 21889.96 at Visit 5) than a booster dose of 15 μg MVC-COV1901 (Subgroup B-1; W+W+W+W; 21078.01 at Visit 4 and 19014.66 at Visit 5).

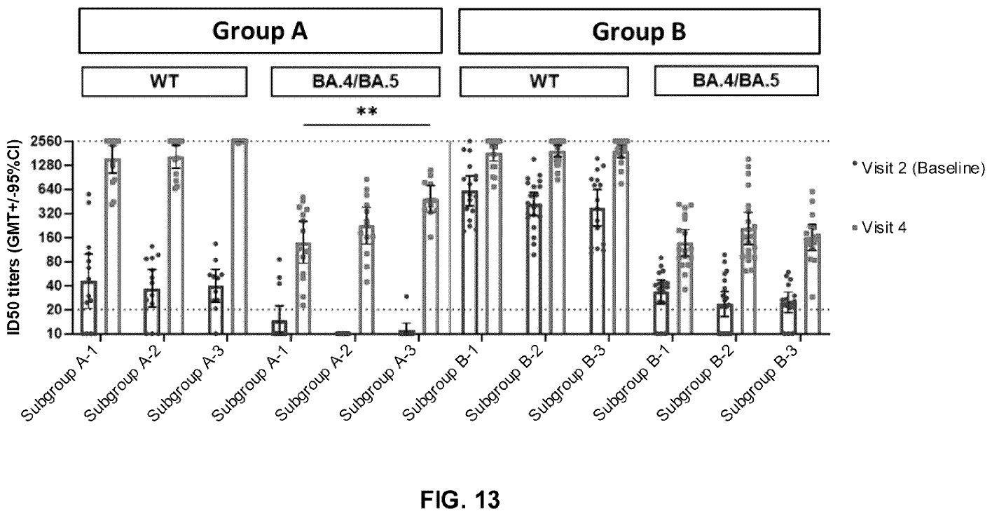

The results of pseudovirus-based neutralization assay are summarized in FIG. 13. In both Groups A and B, all types of booster dose elicited uniformly high levels of neutralizing antibody against WT pseudovirus. In Group A, at Visit 4, a booster dose of 25 μg MVC-COV1901-Beta (Subgroup A-3; W+W+25B) elicited the highest level of neutralizing antibody against the Omicron variant pseudovirus (ID₅₀ GMT: 425.65). In addition, a booster dose of 15 μg MVC-COV1901-Beta (Subgroup A-2; W+W+15B) elicited a higher level of neutralizing antibody against the Omicron variant pseudovirus (ID₅₀ GMT: 240.10) than a booster dose of 15 μg MVC-COV1901 (Subgroup A-1; W+W+W; GMT: 139.11). In particular, a booster dose of 25 μg MVC-COV1901-Beta (Subgroup A-3; W+W+25B) induced a significantly higher level of neutralizing antibody against the Omicron variant pseudovirus compared to a booster dose of 15 μg MVC-COV1901 (Subgroup A-1; W+W+W) (p<0.01).

In Group B (FIG. 13), at Visit 4, a booster dose of 15 μg MVC-COV1901-Beta (Subgroup B-2; W+W+W+15B) elicited the highest level of neutralizing antibody against the Omicron variant pseudovirus (ID₅₀ GMT: 222.44). In addition, a booster dose of 25 μg MVC-COV1901-Beta (Subgroup B-3; W+W+W+25B) had a higher level of neutralizing antibody against the Omicron variant pseudovirus (ID₅₀ GMT: 154.62) than a booster dose of 15 μg MVC-COV1901 (Subgroup B-1; W+W+W+W; ID₅₀ GMT: 136.83).

In conclusion, solicited adverse events were mostly mild and similar. After 2 or 3 prior doses of MVC-COV1901, a booster dose of MVC-COV1901-Beta (either 15 μg or 25 μg per dose) induced numerically higher levels of neutralizing antibody against WT, Beta and Omicron variants and anti-Spike IgG than a booster dose of MVC-COV1901.

These results indicate that administration of at least one dose of the Beta recombinant protein in a multiple-dose regimen is safe and provides improved immunogenicity, an enhanced immune response, and/or broad-spectrum immunity.

Many changes and modifications in the above-described embodiment of the invention can, of course, be carried out without departing from the scope thereof.

Accordingly, to promote progress in science and the useful arts, the invention is disclosed and is intended to be limited only by the scope of the appended claims.

What is claimed is:

1. An immunogenic composition against a severe acute respiratory syndrome coronavirus 2 (SARS-COV-2), comprising an antigenic recombinant protein and an adjuvant selected from the group consisting of an aluminum-containing adjuvant, an unmethylated cytosine-phosphate-guanosine (CpG) motif, and a combination thereof, wherein the antigenic recombinant protein substantially consists of residues 14-1205 of spike protein of SARS-COV-2 Beta variant with proline substitutions at residues 983 and 984 and a “GSAS” substitution at residues 679-682 and a C-terminal T4 fibritin trimerization domain.

2. The immunogenic composition of claim 1, wherein the antigenic recombinant protein comprises an amino acid sequence of SEQ ID NO: 14 or 15, or the amino acid sequence at least 95%, 96%, 97%, 98%, or 99% identical to SEQ ID NO: 14 or 15.

3. The immunogenic composition of claim 1, wherein the aluminum-containing adjuvant comprises aluminum hydroxide, aluminum oxyhydroxide, aluminum hydroxide gel, aluminum phosphate, aluminum phosphate gel, aluminum hydroxyphosphate, aluminum hydroxyphosphate sulfate, amorphous aluminum hydroxyphosphate sulfate, potassium aluminum sulfate, aluminum monostearate or a combination thereof.

4. The immunogenic composition of claim 1, wherein a 0.5 ml dose of the immunogenic composition comprises from about 250 to about 1500 μg Al³⁺, or about 375 μg Al³⁺ or about 750 μg Al³⁺.

5. The immunogenic composition of claim 1, wherein the unmethylated CpG motif comprises a synthetic oligodeoxynucleotide (ODN) of SEQ ID NO: 7, SEQ ID NO: 8, SEQ ID NO: 9, SEQ ID NO: 10, SEQ ID NO: 11, SEQ ID NO: 12, or a combination thereof.

6. The immunogenic composition of claim 1, wherein a 0.5 ml dose of the immunogenic composition comprises from about 750 to about 3000 μg of the unmethylated CpG motif, or about 750 μg, 1500 μg, or 3000 μg of the unmethylated CpG motif.

7. The immunogenic composition of claim 1, wherein the immunogenic composition can be stored at 40° C. to 42° C. for 3 to 7 days.

8. A method for eliciting an immune response against a severe acute respiratory syndrome coronavirus 2 (SARS-COV-2) in a subject in need thereof, comprising administering to the subject at least one dose of an immunogenic composition of claim 1.

9. The method of claim 8, wherein the subject is administered a first dose and a second dose of the immunogenic composition of claim 1 with a suitable interval between the first dose and the second dose.

10. The method of claim 8, wherein the subject is administered a first dose, a second dose, and a third dose of the immunogenic composition of claim 1 with a first suitable interval between the first dose and the second dose, and with a second suitable interval between the second dose and the third dose.

11. The method of claim 8, wherein the subject is administered a first dose of an immunogenic composition derived from SARS-COV-2 wild type (WT) strain, and a second dose of the immunogenic composition of claim 1 with a suitable interval between the first dose and the second dose, and wherein the immunogenic composition derived from SARS-COV-2 WT strain comprises a polynucleotide sequence encoding a polypeptide sequence at least 90%, 95%, 96%, 97%, 98%, 99%, or 100% identical to SEQ ID NO: 5 or 6 or a polypeptide sequence at least 90%, 95%, 96%, 97%, 98%, 99%, or 100% identical to SEQ ID NO: 5 or 6.

12. The method of claim 11, wherein the immunogenic composition derived from SARS-COV-2 WT strain comprises a polypeptide sequence at least 90%, 95%, 96%, 97%, 98%, 99%, or 100% identical to SEQ ID NO: 5 or 6, and an adjuvant selected from the group consisting of an aluminum-containing adjuvant, an unmethylated cytosine-phosphate-guanosine (CpG) motif, and a combination thereof.

13. The method of claim 8, wherein the subject is administered a first dose and a second dose of an immunogenic composition derived from SARS-COV-2 WT strain, and a third dose of the immunogenic composition of claim 1 with a first suitable interval between the first dose and the second dose, and with a second suitable interval between the second dose and the third dose, and wherein the immunogenic composition derived from SARS-COV-2 WT strain comprises a polynucleotide sequence encoding a polypeptide sequence at least 90%, 95%, 96%, 97%, 98%, 99%, or 100% identical to SEQ ID NO: 5 or 6 or a polypeptide sequence at least 90%, 95%, 96%, 97%, 98%, 99%, or 100% identical to SEQ ID NO: 5 or 6.

14. The method of claim 13, wherein the immunogenic composition derived from SARS-COV-2 WT strain comprises a polypeptide sequence at least 90%, 95%, 96%, 97%, 98%, 99%, or 100% identical to SEQ ID NO: 5 or 6, and an adjuvant selected from the group consisting of an aluminum-containing adjuvant, an unmethylated cytosine-phosphate-guanosine (CpG) motif, and a combination thereof.

15. The method of claim 8, wherein the subject is administered a first dose, a second dose, and a third dose of an immunogenic composition derived from SARS-COV-2 WT strain, and a fourth dose of the immunogenic composition of claim 1 with a first suitable interval between the first dose and the second dose, a second suitable interval between the second dose and the third dose, and a third suitable interval between the third dose and the fourth dose, and wherein the immunogenic composition derived from SARS-COV-2 WT strain comprises a polynucleotide sequence encoding a polypeptide sequence at least 90%, 95%, 96%, 97%, 98%, 99%, or 100% identical to SEQ ID NO: 5 or 6 or a polypeptide sequence at least 90%, 95%, 96%, 97%, 98%, 99%, or 100% identical to SEQ ID NO: 5 or 6.

16. The method of claim 15, wherein the immunogenic composition derived from SARS-COV-2 WT strain comprises a polypeptide sequence at least 90%, 95%, 96%, 97%, 98%, 99%, or 100% identical to SEQ ID NO: 5 or 6, and an adjuvant selected from the group consisting of an aluminum-containing adjuvant, an unmethylated cytosine-phosphate-guanosine (CpG) motif, and a combination thereof.

17. The method of claim 8, wherein the subject is administered a first dose of an immunogenic composition derived from SARS-COV-2 WT strain, and a second dose and a third dose of the immunogenic composition of claim 1 with a first suitable interval between the first dose and the second dose, and with a second suitable interval between the second dose and the third dose, and wherein the immunogenic composition derived from SARS-COV-2 WT strain comprises a polynucleotide sequence encoding a polypeptide sequence at least 90%, 95%, 96%, 97%, 98%, 99%, or 100% identical to SEQ ID NO: 5 or 6 or a polypeptide sequence at least 90%, 95%, 96%, 97%, 98%, 99%, or 100% identical to SEQ ID NO: 5 or 6.

18. The method of claim 17, wherein the immunogenic composition derived from SARS-COV-2 WT strain comprises a polypeptide sequence at least 90%, 95%, 96%, 97%, 98%, 99%, or 100% identical to SEQ ID NO: 5 or 6, and an adjuvant selected from the group consisting of an aluminum-containing adjuvant, an unmethylated cytosine-phosphate-guanosine (CpG) motif, and a combination thereof.

19. The method of claim 8, wherein the severe acute respiratory syndrome coronavirus 2 (SARS-COV-2) is a WT strain or a variant.

20. The method of claim 8, wherein the immunogenic composition is administered by intramuscular injection.
